(19) United States
(12) Patent Application Publication
(10) Pub. No.: US 2023/0206435 A1
(43) Pub. Date: Jun. 29, 2023

(54) ARTIFICIAL INTELLIGENCE-BASED GASTROSCOPY DIAGNOSIS SUPPORTING SYSTEM AND METHOD FOR IMPROVING GASTROINTESTINAL DISEASE DETECTION RATE

(71) Applicant: INFINITT HEALTHCARE CO., LTD., Seoul (KR)

(72) Inventors: So Hyun BYUN, Seoul (KR); Hyun Ji CHOI, Seoul (KR); Chung Il AHN, Seoul (KR)

(21) Appl. No.: 18/087,945

(22) Filed: Dec. 23, 2022

(30) Foreign Application Priority Data
Dec. 24, 2021 (KR) 1020210187355

(51) Int. Cl. G06T 7/00; G16H 30/40

(52) U.S. Cl. CPC G06T 7/0012; G16H 30/40; G06T 2207/20081; G06T 2207/30092

(57) ABSTRACT

A gastroscopic image diagnosis supporting system is configured to analyze a video frame of the gastroscopic image using a first medical image analysis model of the medical image analysis models; classify a gastrointestinal anatomical position of the video frame by identifying a part corresponding to a preset gastrointestinal anatomical position class in the video frame; and store information about the gastrointestinal anatomical position of the video frame as index information of the finding information together with finding information when a user captures and stores the video frame as the finding information about a gastrointestinal lesion.

### CROSS-REFERENCE TO RELATED APPLICATION

This application claims priority from Korean Application No. 10-2021-0187355 filed on Dec. 24, 2021, which is incorporated herein by reference in its entirety.

### TECHNICAL FIELD

The present disclosure relates generally to an automatic medical image diagnosis support apparatus and method, and more particularly to an artificial intelligence-based medical image analysis software, system, and method assisting in diagnosing gastric endoscopy images during gastroscopy and assisting in reducing the risk of missing lesions.

### RELATED ART

Endoscopic diagnosis is a medical practice that is considerably frequently performed for the purpose of regular medical examination. There is a demand for a technology that processes real-time images upon endoscopic diagnosis to preprocess them so that an expert can easily identify a lesion at a medical site. Recently, U.S. Patent Application Publication No. US 2018/0253839 entitled “A System and Method for Detection of Suspicious Tissue Regions in an Endoscopic Procedure” introduced a technology that performed a preprocessing process of removing noise from an image frame and performed a noise removal preprocessing process and a computer-aided diagnosis (CAD) process in parallel, thereby providing real-time diagnosis assistance display information.

In this technology, the accuracy and reliability of a CAD module are recognized as significantly important factors.

Technologies for segmenting or detecting objects in an image or classifying objects in an image are used for various purposes in image processing. In a medical image, objects in the image are segmented, detected, and classified based on the brightness or intensity values of the image, in which case each of the objects may be an organ of the human body, or a lesion.

Recently, the introduction of deep learning and a convolutional neural network (CNN) as artificial neural networks into the automation of an image processing process has dramatically improved the performance of an automated image processing process.

However, on the other hand, the insides of recent artificial neural networks, such as deep learning and a CNN, approximate black boxes, and thus there is reluctance for a user to fully accept and adopt them even when acquired results are excellent. In particular, reluctance to artificial neural networks stands out as being more important in the medical imaging field in which human life is dealt with.

Under this background, research into explainable artificial intelligence (X-AI) has been attempted in the Defense Advanced Research and Planning (DARPA) of the U.S., etc. (see https://www.darpa.mil/program/explainable-artificial-intellig-ence). However, no visible results have yet been revealed.

In the case of using artificial intelligence, especially artificial neural networks, in the field of medical imaging, there is a problem in that it is difficult for clinicians to have confidence in whether these artificial intelligence techniques are clinically useful because it is not possible to derive descriptive information (explanation) about the process by which results are obtained.

A similar problem is still present in a medical image diagnosis process in that it is difficult to have clinical confidence in a process in which an artificial intelligence diagnosis system that operates like a black box generates a result.

In a known previous research (S. Kumar et al., “Adenoma miss rates associated with a 3 minute versus 6 minute colonoscopy withdrawal time: a prospective, randomized trial”), it is known that a maximum of 25% of lesions may be missed during gastroscopy. This phenomenon is known to occur due to a problem with an image, a blind spot, or human error. Due to successive and repetitive procedures, doctors often exhibit signs of fatigue, so that lesions may not be sufficiently detected. Therefore, human error may cause the missing of lesions, which negatively affects the medical results of examinations.

Korean Patent No. 10-2255311 entitled “Artificial Intelligence-based Gastroscopic Image Analysis Method” discloses a process of implementing artificial intelligence-based multiple image classification models for gastroscopic images and training data for training the image classification models. According to Korean Patent No. 10-2255311, there are disclosed a technology for automatically classifying and recognizing the anatomical positions of the stomach using the image classification models and a technology for automatically storing the positions of lesions.

Korean Patent Application Publication No. 10-2020-0038120 entitled “Apparatus and Method for Diagnosing Gastric Lesions Using Deep Learning of Gastroscopic Images” discloses a technology for detecting a lesion in an image obtained via a gastroscope and classifying the category of the lesion, i.e., determining whether the lesion is a tumor or a non-tumor.

Therefore, there is a need for a more intuitive and convenient user interface that supports the reading of a user, who is a medical professional, using the reading information of an artificial neural network obtained via the prior art.

### SUMMARY

An object of the present invention is to generate and provide an optimized combination of a plurality of artificial intelligence medical image diagnosis results as display information for each real-time image frame.

An object of the present invention is to provide an optimized combination of a plurality of artificial intelligence medical image diagnosis results capable of efficiently displaying diagnosis results that are likely to be acquired, are likely to be overlooked, or have a high level of risk in a current image frame.

An object of the present invention is to provide a user interface and diagnosis computing system that automatically detect and present diagnosis results that are likely to be acquired, are likely to be overlooked, or have a high level of risk in a current image frame, so that medical staff can check and review the diagnosis results in real time during an endoscopy.

An object of the present invention is to train on polyps, ulcers, various gastric diseases, etc., which may be missed by a user, based on artificial intelligence medical image diagnosis/diagnosis results for each real-time video frame of a gastroscopic image via an artificial intelligence algorithm and then apply the results of the training to an artificial intelligence diagnosis assisting system, thereby increasing work efficiency and diagnostic accuracy.

An object of the present invention is to automatically detect a disease that may easily be missed by a user during gastroscopy and present the location of the disease in a gastric path (a gastroscopic path), so that the user may easily check the disease in real time during gastroscopy and even a report adapted to enable other examiners to check it later may be generated through a simple operation.

In a diagnosis assisting technology for gastroscopic images, which is an application target of the present invention, when various lesions are located together with the stomach wall or folds in the stomach, it is easy to miss lesions when the lesions are not different in color from surrounding tissues and have small sizes. Accordingly, an object of the present invention is to provide a method that may further improve disease detection rate in the stomach by detecting various lesions in real time via artificial intelligence and also to help other examiners to check lesions again later by providing the locations of the lesions in a gastroscopic path.

An object of the present invention is to display a blind spot, which is a missing part not checked by examination, to be easily identified by accumulating and displaying the anatomical positions of parts examined by a gastroscope in real time during gastroscopy, to increase the adenoma detection rate by detecting various lesions in the stomach regardless of size and color, and to help a user, who is a medical professional, to easily identify the position of each lesion in the process of performing subsequent examination or reviewing examination results after the completion of examination by also displaying the anatomical position of the corresponding lesion.

Another object of the present invention is to provide a user interface that also displays pictures, captured and/or stored as finding information by an examiner during gastroscopy, after the termination of the gastroscopy in accordance with the anatomical positions thereof in the stomach, thereby helping a user to search for or access finding information more rapidly and conveniently when making follow-up decisions by using examination results.

According to an aspect of the present invention, there is provided a gastroscopic image diagnosis supporting system for supporting diagnosis of a medical image, the gastroscopic image diagnosis supporting system comprising a computing system. The computing system comprises: a reception interface configured to receive a gastroscopic image as the medical image; memory or a database configured to store one or more medical image analysis models each having a function of analyzing the gastroscopic image; and a processor.

The processor is configured to: analyze a video frame of the gastroscopic image using a first medical image analysis model of the medical image analysis models; classify a gastrointestinal anatomical position of the video frame by identifying a part corresponding to a preset gastrointestinal anatomical position class in the video frame; and store information about the gastrointestinal anatomical position of the video frame as index information of the finding information together with finding information when a user captures and stores the video frame as the finding information about a gastrointestinal lesion.

The processor may be further configured to: provide a gastrointestinal anatomical position map; display the gastrointestinal anatomical position of the video frame on the gastrointestinal anatomical position map and provide the gastrointestinal anatomical position map together with the video frame to the user while the video frame is displayed to the user through a user display; and display a gastrointestinal anatomical position of the finding information on the gastrointestinal anatomical position map and provide the gastrointestinal anatomical position map to the user when the user requests checking of the finding information after gastroscopy.

The processor may be further configured to: analyze the video frame of the gastroscopic image by using a second medical image analysis model of the medical image analysis models; detect whether a region suspected of being a lesion is present in the video frame; and classify a type of lesion by comparing the region suspected of being a lesion against lesion classes by using a third medical image analysis model of the medical image analysis models.

The processor may be further configured to: generate gastroscopy entry and exit path information based on the information about the gastrointestinal anatomical position of the video frame and a sequential position at which the video frame is acquired; and provide the gastroscopy entry and exit path information to the user by displaying the gastroscopy entry and exit path information on a gastrointestinal anatomical position map through a user display.

The processor may be further configured to: display a user interface for accessing finding information of previous gastroscopy on a first gastrointestinal anatomical position map at a gastrointestinal anatomical position of the finding information of the previous gastroscopy; provide the first gastrointestinal anatomical position map to the user; display a path of current gastroscopy and a gastrointestinal anatomical position of a current video frame on a second gastrointestinal anatomical position map; and provide the second gastrointestinal anatomical position map to the user.

According to another aspect of the present invention, there is provided a gastroscopic image diagnosis supporting system for supporting diagnosis of a medical image, the gastroscopic image diagnosis supporting system comprising a computing system comprises a reception interface; memory or a database configured to store one or more medical image analysis models each having a function of analyzing the gastroscopic image; and a processor.

The processor is configured to: analyze a video frame of the gastroscopic image by using a first medical image analysis model of the medical image analysis models; classify a gastrointestinal anatomical position of the video frame by identifying a part corresponding to a preset gastrointestinal anatomical position class in the video frame; display whether a lesion has been found at a position corresponding to the anatomical position class and statistical information about one or more lesions found at the position corresponding to the anatomical position class on a gastrointestinal anatomical position map; and provide the gastrointestinal anatomical position map to a user through a user display.

The processor may be further configured to: display whether a lesion has been found at the position corresponding to the anatomical position class and the statistical information about one or more lesions found at the position corresponding to the anatomical position class on a first gastrointestinal anatomical position map by using an analysis result of a video frame of previous gastroscopy; and display a path of current gastroscopy and a gastrointestinal anatomical position of a current video frame on a second gastrointestinal anatomical position map by using an analysis result of a video frame of the current gastroscopy.

The processor may be further configured to display whether a missing anatomical position class is present in the video frame of the previous gastroscopy and an anatomical position of the missing anatomical position class on the first gastrointestinal anatomical position map by using the analysis result of the video frame of the previous gastroscopy.

According to another aspect of the present invention, there is provided a gastroscopic image diagnosis supporting system for supporting diagnosis of a medical image, the gastroscopic image diagnosis supporting system comprising a computing system comprises a reception interface; memory or a database configured to store one or more medical image analysis models each having a function of analyzing the gastroscopic image; and a processor.

The processor is configured to: analyze a video frame of the gastroscopic image using a first medical image analysis model of the medical image analysis models; classify a gastrointestinal anatomical position of the video frame by identifying a part corresponding to a preset gastrointestinal anatomical position class in the video frame; generate gastroscopy entry and exit path information based on information about the gastrointestinal anatomical position of the video frame and a sequential position at which the video frame is acquired; and store the information about the gastrointestinal anatomical position of the video frame and the gastroscopy entry and exit path information as index information of the video frame together with the video frame.

The processor may be further configured to: provide a gastrointestinal anatomical position map; display a user interface for accessing the video frame on the gastrointestinal anatomical position map at the gastrointestinal anatomical position of the video frame; and provide the gastrointestinal anatomical position map to a user.

The processor may be further configured to: provide a gastrointestinal anatomical position map; display whether the video frame is present at a position corresponding to the anatomical position class, whether a missing anatomical position class is present in the video frame of the gastroscopy, and an anatomical position of the missing anatomical position class on the gastrointestinal anatomical position map; and provide the gastrointestinal anatomical position map to a user.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing a gastroscopic image diagnosis assisting system having a multi-client structure and peripheral devices according to an embodiment of the present invention;

FIG. 2 is a diagram showing a gastroscopic image diagnosis assisting system having a single client structure and peripheral devices according to an embodiment of the present invention;

FIG. 3 is a diagram showing the workflow of a gastroscopic image diagnosis assisting system according to an embodiment of the present invention;

FIG. 4 shows views illustrating examples of an image in which a gastroscopic image and display information are displayed together according to an embodiment of the present invention;

FIG. 5 is an example of a process of classifying an anatomical position in the stomach during gastroscopy performed by a gastroscopic image diagnosis supporting system according to an embodiment of the present invention;

FIG. 6 is an example of display information and/or a user interface that are provided together with a gastroscopic image by the gastroscopic image diagnosis supporting system according to the embodiment of the present invention;

FIG. 7 is an example of a process of detecting a lesion in a gastroscopic image and classifying an anatomical position that is performed by the gastroscopic image diagnosis supporting system according to the embodiment of the present invention;

FIG. 8 shows an example of a user interface provided by the gastroscopic image diagnosis supporting system according to the embodiment of the present invention, and is a diagram showing a user interface for lesion findings displayed together with a gastrointestinal anatomical position map during or after examination and a process of calling findings;

FIG. 9 is an operational flowchart showing the workflow of a method performed by the gastroscopic image diagnosis supporting system according to the embodiment of the present invention; and

FIG. 10 is an example of display information and/or a user interface that are provided together with a gastroscopic image by the gastroscopic image diagnosis supporting system according to the embodiment of the present invention.

### DETAILED DESCRIPTION

Exemplary embodiments of the present disclosure are disclosed herein. However, specific structural and functional details disclosed herein are merely representative for purposes of describing exemplary embodiments of the present disclosure. Thus, exemplary embodiments of the present disclosure may be embodied in many alternate forms and should not be construed as limited to exemplary embodiments of the present disclosure set forth herein.

Accordingly, while the present disclosure is capable of various modifications and alternative forms, specific exemplary embodiments thereof are shown by way of example in the drawings and will herein be described in detail. It should be understood, however, that there is no intent to limit the present disclosure to the particular forms disclosed, but on the contrary, the present disclosure is to cover all modifications, equivalents, and alternatives falling within the spirit and scope of the present disclosure. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that “at least one of A and B” may be used herein to indicate “at least one from among a combination of at least one of A and B” or “at least one of A or B”.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between,” “adjacent” versus “directly adjacent,” etc.).

The terminology used herein is for the purpose of describing particular exemplary embodiments only and is not intended to be limiting of the present disclosure. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

When recently rapidly developed deep learning/CNN-based artificial neural network technology is applied to the imaging field, it may be used to identify visual elements that are difficult to identify with the unaided human eye. The application of this technology is expected to expand to various fields such as security, medical imaging, and non-destructive inspection.

For example, in the medical imaging field, there are cases where cancer tissue is not immediately diagnosed as cancer during a biopsy but is diagnosed as cancer after being tracked and monitored from a pathological point of view. Although it is difficult for the human eye to confirm whether or not corresponding cells are cancer in a medical image, there is an expectation that the artificial neural network technology can provide a more accurate prediction than the human eye.

However, although the artificial neural network technology can yield better prediction/classification/diagnosis results than the human eye in some studies, there is a lack of descriptive information about prediction/classification/diagnosis results acquired through the application of the artificial neural network technology, and thus a problem arises in that it is difficult to accept and adopt the above results in the medical field.

The present invention has been conceived from the intention to improve the performance of the classifying/predicting objects in an image, which are difficult to classify with the unaided human eye, through the application of the artificial neural network technology. Furthermore, even in order to improve the classification/prediction performance of the artificial neural network technology, it is significantly important to acquire descriptive information about the internal operation that reaches the generation of a final diagnosis result based on the classification/prediction processes of the artificial neural network technology.

For example, in the field of medical imaging, there are cases where cancer tissue is not immediately diagnosed as cancer during a biopsy but is diagnosed as cancer after being tracked and monitored from a pathological point of view. Although it is difficult for the human eye to confirm whether corresponding cells are cancer in a medical image, there is an expectation that artificial neural network technology can provide a more accurate prediction than the human eye.

However, although the artificial neural network technology can yield better prediction/classification/diagnosis results than the human eye in some studies, the possibility that the artificial neural network technology is utilized is limited by the fact that some part of the artificial neural network technology corresponds to a black-box in which a user cannot know whether the prediction/classification/diagnosis results obtained by applying the artificial neural network technology exhibit high performance by chance or whether they have undergone an appropriate determination process for a target task. In other words, there is a lack of descriptive information about the process by which the analysis results of the artificial neural network technology are obtained, so that a problem arises in that it is difficult to accept and adopt the above results in the medical field.

In contrast, the utilization of a rule-based method is limited in that it is not possible to obtain results as good as deep learning by training or learning in the rule-based method that is easy to describe. Accordingly, research into deep learning-based artificial intelligence that can provide descriptive information (explanation) as well as improved performance is actively conducted. In the practical application of image processing using an artificial neural network, especially in the field of medical imaging, descriptive information about the basis of diagnosis and classification is required. Such descriptive information has not yet been derived based on prior art.

The present invention has been conceived from the intention to improve the performance of the classifying/predicting objects in an image, which are difficult to classify with the unaided human eye, through the application of the artificial neural network technology. Furthermore, even in order to improve the classification/prediction performance of the artificial neural network technology, it is significantly important to acquire descriptive information about the internal operation that reaches the generation of a final diagnosis result based on the classification/prediction processes of the artificial neural network technology. In addition, according to this trend, users who are medical professionals need an intuitive and convenient user interface to more conveniently use the analysis results derived by an artificial neural network, to check for errors, and to additionally search for or utilize necessary information.

Korean Patent No. 10-2255311 entitled “Artificial Intelligence-based Gastroscopic Image Analysis Method” discloses a process of implementing artificial intelligence-based multiple image classification models for gastroscopic images and training data for training the image classification models. According to Korean Patent No. 10-2255311, there are disclosed a technology for automatically classifying and recognizing the anatomical positions of the stomach using the image classification models and a technology for automatically storing the positions of lesions.

Korean Patent Application Publication No. 10-2020-0038120 entitled “Apparatus and Method for Diagnosing Gastric Lesions Using Deep Learning of Gastroscopic Images” discloses a technology for detecting a lesion in an image obtained via a gastroscope and classifying the category of the lesion, i.e., determining whether the lesion is a tumor or a non-tumor.

In the present invention, data storage means, calculation means, the basic concepts and structure of an artificial neural network, transmission/reception interfaces for the transmission of input data (images), etc., which are readily apparent from these prior art documents and the conventional technologies in the relevant arts, are required to implement the invention. However, detailed descriptions of these basic items may obscure the subject matter of the present invention. Accordingly, among the components of the present invention, items known to those of ordinary skill in the art prior to filing the present invention are described as parts of the configuration of the present invention in this specification, and descriptions of items obvious to those of ordinary skill in the art are omitted when it is determined that the detailed descriptions may obscure the subject matter of the invention.

In addition, descriptions of the items omitted therein may be replaced by providing notification that the items are known to those of ordinary skill in the art via the related art documents, e.g., U.S. Patent Application Publication No. US 2018/0253839 entitled “A System and Method for Detection of Suspicious Tissue Regions in an Endoscopic Procedure,” Korean Patent No. 10-2255311 entitled “Artificial Intelligence-based Gastroscopic Image Analysis Method,” and Korean Patent Application Publication No. 10-2020-0038120 entitled “Apparatus and Method for Diagnosing Gastric Lesions Using Deep Learning of Gastroscopic Images,” that are cited therein.

Furthermore, the technologies described in the prior art documents may be included in at least part of the configuration of the present invention within the scope that is consistent with the purpose of the present invention.

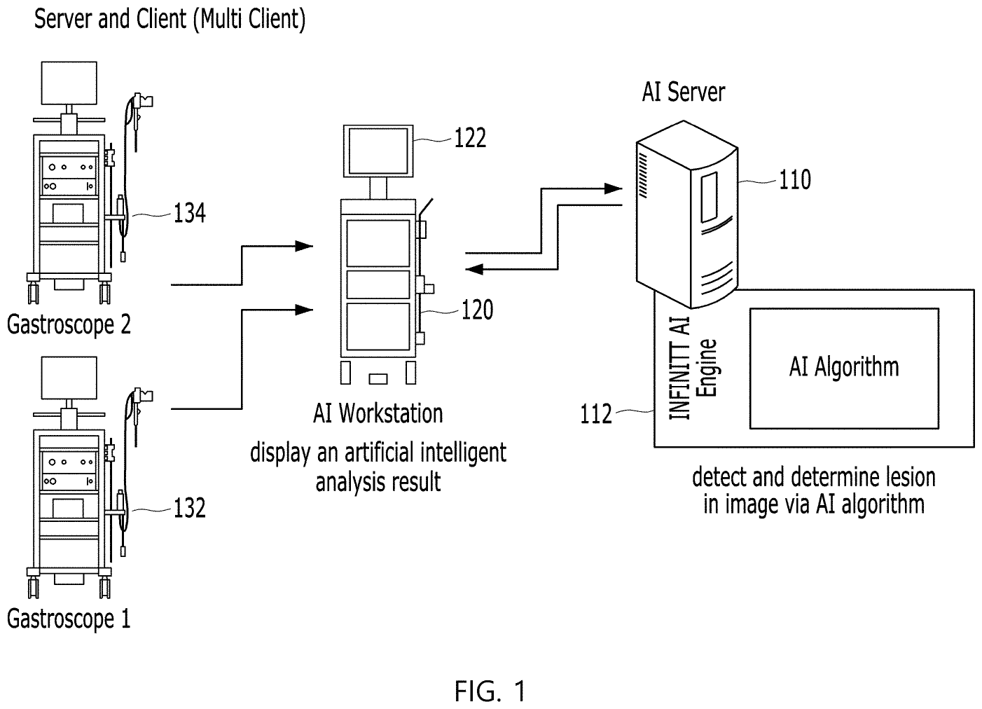

A medical image diagnosis supporting apparatus, system, and method according to embodiments of the present invention will be described in detail below with reference to FIGS. 1 to 10.

FIG. 1 is a diagram showing a gastroscopic image diagnosis assisting system having a multi-client structure and peripheral devices according to an embodiment of the present invention.

A first gastroscopic image acquisition module 132 may transfer a gastroscopic image, acquired in real time, to an artificial intelligence workstation 120 in real time, or may transfer a captured image of a gastroscopic image to the artificial intelligence workstation 120.

A second gastroscopic image acquisition module 134 may transfer a gastroscopic image, acquired in real time, to the artificial intelligence workstation 120 in real time, or may transfer a captured image of a gastroscopic image to the artificial intelligence workstation 120.

In this case, the artificial intelligence workstation 120 may include an input/reception interface module (not shown) configured to receive a gastroscopic image (or a captured image) received from the first gastroscopic image acquisition module 132 and the second gastroscopic image acquisition module 134.

The artificial intelligence workstation 120 may transfer a video frame of a received/acquired gastroscopic image to an artificial intelligence server 110. In this case, the image transferred to the artificial intelligence server 110 may be transferred in a standardized JPEG format or MPEG format. The artificial intelligence server 110 may also include an input/reception interface module (not shown) configured to receive a video frame image in a standardized format.

The artificial intelligence server 110 may detect and determine a lesion in a video frame/image by using an artificial intelligence algorithm (a medical image analysis algorithm) 112.

A processor (not shown) in the artificial intelligence server 110 may input given image data to the artificial intelligence algorithm 112, may receive the analysis result of the artificial intelligence algorithm 112, and may control a data transfer process between memory or storage (not shown) having the artificial intelligence algorithm 112 stored therein and the processor during the above process.

The output interface module (not shown) of the artificial intelligence server 110 may transfer the analysis result of the artificial intelligence algorithm 112 to the artificial intelligence workstation 120. In this case, the transferred information may include whether a finding suspected of being a lesion is detected within a video frame, the coordinates at which the finding suspected of being a lesion is detected, the probability that the finding suspected of being a lesion is a lesion, and the location of the finding suspected of being a lesion in a gastric or gastroscopic path.

The artificial intelligence workstation 120 may display the analysis result of the artificial intelligence algorithm 112 on a user display 122. In this case, the information displayed on the user display 122 may include whether a finding suspected of being a lesion is detected within a video frame of a currently displayed gastroscopic image, the finding suspected of being a lesion that is visualized to be visually distinguished within the video frame (e.g., the finding suspected of being a lesion that is highlighted, or the finding suspected of being a lesion that is surrounded with a box), the coordinates of the location of the finding suspected of being a lesion within the video frame, and the location of the finding suspected of being a lesion in a gastric or gastroscopic path.

In this case, a plurality of gastroscopic images may be simultaneously displayed on the user display 122 in real time. Individual gastroscopic image video frames may be displayed for respective windows on a screen.

In real-time examination, when a user immediately detects a finding suspected of being a lesion on a gastroscopic image and takes action, there is no significant problem. In contrast, when a user misses a finding suspected of being a lesion in a real-time examination, it is substantially impossible to re-diagnose the missing finding suspected of being a lesion in the related arts.

Meanwhile, the present invention has advantages in that a captured video frame may be re-checked by other users later, in that even when an endoscope has already advanced, a previous video frame may be recalled and the missing finding suspected of being a lesion may be re-diagnosed, and in that even after endoscopy has been finished, the coordinates of the location of a finding suspected of being a lesion within the video frame and the location of the finding suspected of being a lesion in a gastroscopic path are provided together, and thus the location of the finding suspected of being a lesion may be identified, so that follow-up actions may be taken.

Although FIG. 1 shows an embodiment in which the artificial intelligence workstation 120 and the artificial intelligence server 110 are separated from each other for convenience of description, this is merely an embodiment of the present invention. However, it will be apparent to those of ordinary skill in the art that according to another embodiment of the present invention, the artificial intelligence workstation 120 and the artificial intelligence server 110 may be implemented to be combined together in a single computing system.

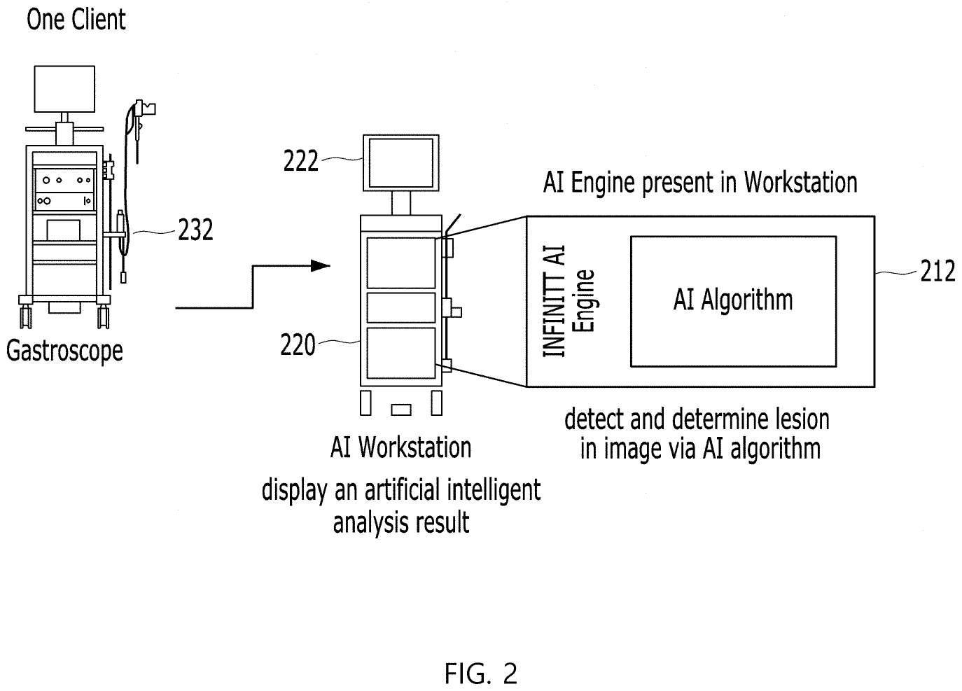

FIG. 2 is a diagram showing a gastroscopic image diagnosis assisting system having a single client structure and peripheral devices according to an embodiment of the present invention.

A gastroscopic image acquisition module 232 may transfer a gastroscopic image, acquired in real time, to an artificial intelligence workstation 220 in real time, or may transfer a captured image of a gastroscopic image to the artificial intelligence workstation 220.

In this case, the artificial intelligence workstation 220 may include an input/reception interface module (not shown) configured to receive a gastroscopic image (or a captured image) received from the gastroscopic image acquisition module 232.

The artificial intelligence workstation 220 may detect and determine a lesion in a video frame/image by using an artificial intelligence algorithm (a medical image analysis algorithm) 212.

A processor (not shown) in the artificial intelligence workstation 220 may input given image data to the artificial intelligence algorithm 212, may receive the analysis result of the artificial intelligence algorithm 212, and may control a data transfer process between memory or storage (not shown) having the artificial intelligence algorithm 212 stored therein and the processor during the above process.

The output interface module (not shown) of the artificial intelligence workstation 220 may generate the analysis result of the artificial intelligence algorithm 212 as display information, and may transfer the display information to the user display 222. In this case, the transferred information may include whether a finding suspected of being a lesion is detected within a video frame, the coordinates at which the finding suspected of being a lesion is detected, the probability that the finding suspected of being a lesion is a lesion, and the location of the finding suspected of being a lesion in a gastric or gastroscopic path.

The artificial intelligence workstation 220 may display the analysis result of the artificial intelligence algorithm 212 on a user display 222. In this case, the information displayed on the user display 222 may include whether a finding suspected of being a lesion is detected within a video frame of a currently displayed gastroscopic image, the finding suspected of being a lesion that is visualized to be visually distinguished within the video frame (e.g., the finding suspected of being a lesion that is highlighted, or the finding suspected of being a lesion that is surrounded with a box), the coordinates of the location of the finding suspected of being a lesion within the video frame, and the location of the finding suspected of being a lesion in a gastric or gastroscopic path.

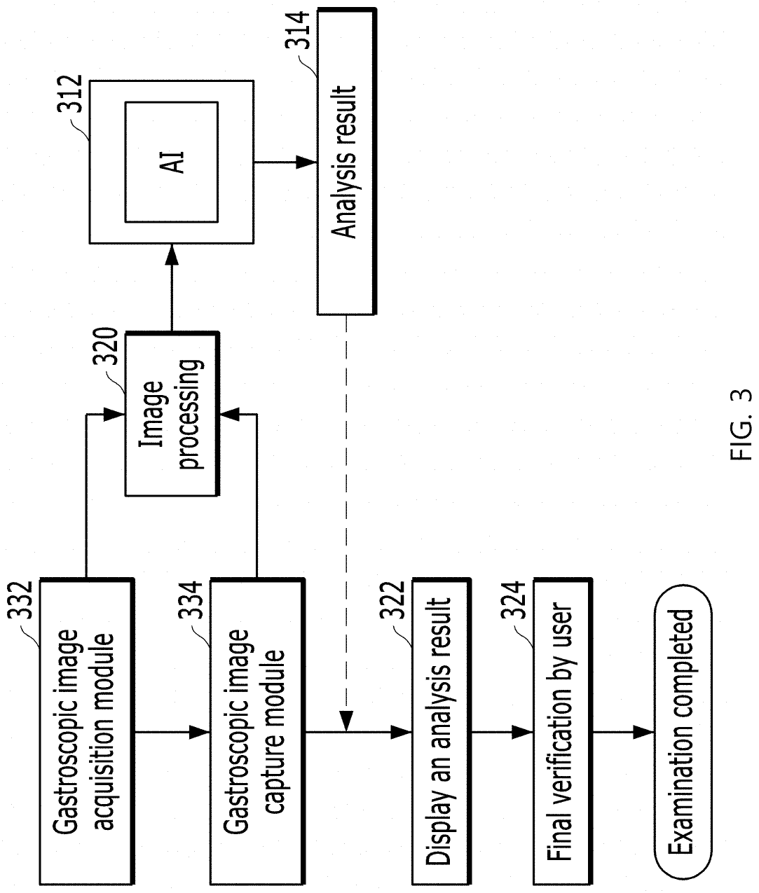

FIG. 3 is a diagram showing the workflow of a gastroscopic image diagnosis assisting system according to an embodiment of the present invention.

The gastroscopic image diagnosis assisting system according to the present embodiment includes a computing system, and the computing system includes: a receiving interface; memory or a database; a processor; and a user display. The receiving interface receives or receives a medical image, and the memory or database stores at least one medical image analysis algorithm 312 having a function of diagnosing a medical image (a gastroscopic image).

A gastroscopic image acquisition module 332 may transfer a gastroscopic image, acquired in real time, to the gastroscopic image diagnosis assisting system, or a gastroscopic image capture module 334 may capture a gastroscopic image and transfer the captured gastroscopic image to the gastroscopic image diagnosis assisting system.

The processor may perform image processing 320 including cropping adapted to remove the black border portions of a gastroscopic image and/or a captured image, rotation/tilting, and the correction of image brightness values.

The processor analyzes a video frame of a gastroscopic image using at least one medical image analysis algorithm 312, detects a finding suspected of being a lesion, whether the finding suspected of being a lesion is present in the video frame, calculates the coordinates of the location of a finding suspected of being a lesion when the finding suspected of being a lesion is present in the video frame, and generates an analysis result 314 including whether a finding suspected of being a lesion is present and the coordinates of the location of the finding suspected of being a lesion. The processor generates display information to be displayed together with the gastroscopic image based on the analysis result 314.

The user display displays the analysis result 314 together with the gastroscopic image (see 322). In other words, when a finding suspected of being a lesion is present in the video frame, the user display displays the finding suspected of being a lesion so that it is visually distinguished on the video frame (see 322) based on the display information, and also displays the coordinates of the location of the finding suspected of being a lesion so that they are visually associated with the finding suspected of being a lesion (see 322).

The processor may calculate the location of a finding suspected of being a lesion in a gastroscopic path, and may generate display information, including whether the finding suspected of being a lesion is present, the coordinates of the location of the finding suspected of being a lesion, and the location of the finding suspected of a lesion in the gastroscopic path. In this case, the processor may calculate the location of the finding suspected of a lesion in the gastroscopic path based on the information of a sensor on a gastroscopic device and/or the analysis result 314 of the artificial intelligence algorithm 312.

The user display may display the location of the finding suspected of being a lesion in the gastroscopic path so that it is visually associated with the finding suspected of being a lesion based on display information (see 322).

The processor may track the location of a video frame, indicative of a current examination region, in the gastroscopic path, and may calculate the location of the finding suspected of being a lesion in the gastroscopic path based on the location of the video frame in the gastroscopic path and the coordinates of the location of the finding suspected of being a lesion.

The processor may calculate the location of the finding suspected of being a lesion in the gastroscopic path based on a pre-examination medical image including the three-dimensional (3D) anatomical structure of a patient to be examined.

A user may finally verify the display information displayed together with the endoscopic image (see 324), may accept or reject the finding suspected of being a lesion, included in the display information, as a lesion, and may, when the finding suspected of being a lesion is accepted as a lesion, take subsequent actions for the lesion or prepare a report in order to take subsequent actions later, thereby causing gastroscopy to be terminated.

The artificial intelligence-based medical image (gastroscopic image) analysis algorithm 312 may be trained using a label, including an indication of a detected lesion for each video frame, the coordinates of the location of the lesion in the video frame, and the location of the lesion in a gastroscopic path, together with each video frame as training data. Accordingly, the processor may calculate the location of the finding suspected of being a lesion in the video frame in the gastroscopic path by using the medical image analysis algorithm 312 and may provide it as the analysis result 314.

In an embodiment of the present invention, a main means for identifying a current location in a path indicated by an endoscopic image may mainly depend on learning and inference regarding endoscopic images.

In this case, when a current location in a gastroscopic path is identified depending on learning and reasoning regarding endoscopic images, the label of each endoscopic image used for learning may include information about a location in an endoscopic (gastroscopic) path and information about a lesion detected/verified (an image actually verified through biopsy) separately for each frame.

In another embodiment of the present invention, learning and inference regarding endoscopic images is a main means for identifying a current location in a gastric path, and additionally a current location may be more accurately identified by combining the main means with an additional means for identifying the current location by estimating the progress speed of frames of an endoscopic image through image analysis.

Furthermore, in general, it is difficult to take a CT image before endoscopy, so that it is necessary to identify a current location in a gastric path by relying only on an endoscopic image. However, if it is possible to take a CT image before endoscopy, a current location may be identified in association with a 3D model of an endoscopic target (the stomach) reconstructed based on a CT image taken before endoscopy in another embodiment of the present invention.

In this case, a CT image-based 3D model of the stomach may be implemented in combination with virtual endoscopic imaging technology, which corresponds to the patent issued to the present applicant (Korean Patent No. 10-1850385 or 10-1230871).

Furthermore, according to another embodiment of the present invention, when a current location in an endoscopic path within an endoscopic examination target (the stomach or the colon) is identified, correction (compensation) may be performed in association with an endoscope or a sensor installed in an endoscopic device (a sensor capable of detecting the length of an endoscope inserted into the human body) rather than relying solely on image analysis.

According to an embodiment of the present invention, the receiving interface may receive at least one endoscopic image from at least one endoscopic image acquisition module. In this case, the processor may detect a finding suspected of being a lesion, whether the finding suspected of being a lesion is present in each video frame of at least one endoscopic image by using at least one medical image analysis algorithm 312. The processor may generate display information including whether a finding suspected of being a lesion is present and the coordinates of the location of the finding suspected of being a lesion for each video frame of at least one gastroscopic image.

A medical image diagnosis assisting method according to another embodiment of the present invention is performed by a processor in a diagnosis assisting system (a computing system) that assists the diagnosis of medical images, and is performed based on program instructions that are loaded into the processor.

A gastroscopic image diagnosis assisting method according to an embodiment of the present invention is performed by the gastroscopic image diagnosis assisting system including the processor and the user display, and may utilize the at least one medical image analysis algorithm having a gastroscopic analysis function stored in the memory or the database included in the gastroscopic image diagnosis assisting system.

The method of the present invention includes the steps of: receiving a gastroscopic image; analyzing, by the processor, each video frame of the gastroscopic image by using the at least one medical image analysis algorithm, and detecting, by the processor, whether a finding suspected of being a lesion is present in the video frame; calculating, by the processor, the coordinates of the location of a finding suspected of being a lesion when the finding suspected of being a lesion is present in the video frame; generating, by the processor, display information including whether a finding suspected of being a lesion is present and the coordinates of the location of the finding suspected of being a lesion; when the finding suspected of being a lesion is present in the video frame, displaying, by the user display, the finding suspected of being a lesion on the video frame so that it is visually distinguished in the video frame based on the display information; and displaying, by the user display, the coordinates of the location of the finding suspected of being a lesion so that they are visually associated with the finding suspected of being a lesion.

In this case, the method of the present invention may further include the step of calculating, by the processor, the location of a finding suspected of being a lesion in a gastroscopic path.

In the method of the present invention, the step of generating the display information includes the step of generating, by the processor, display information, including whether a finding suspected of being a lesion, the coordinates of the location of the finding suspected of being a lesion, and the location of the finding suspected of being a lesion in the gastroscopic path.

In the method of the present invention, the step of displaying, by the user display, the coordinates of the location of the finding suspected of being a lesion so that they are visually associated with the finding suspected of being a lesion includes the step of displaying, by the user display, the location of the finding suspected of being a lesion in the gastroscopic path so that it is visually associated with the finding suspected of being a lesion.

In the method of the present invention, the step of receiving the gastroscopic image may include the step of receiving at least one gastroscopic image from at least one gastroscopic image acquisition module.

In the method of the present invention, the step of detecting whether a finding suspected of being a lesion is present in the video frame includes the step of detecting whether a finding suspected of being a lesion is present in each video frame of the at least one endoscopic image by using the at least one medical image analysis algorithm.

In the method of the present invention, the step of generating the display information includes the step of generating display information, including information about whether a finding suspected of being a lesion is present for each video frame of the at least one endoscopic image and the coordinates of the location of the finding suspected of being a lesion.

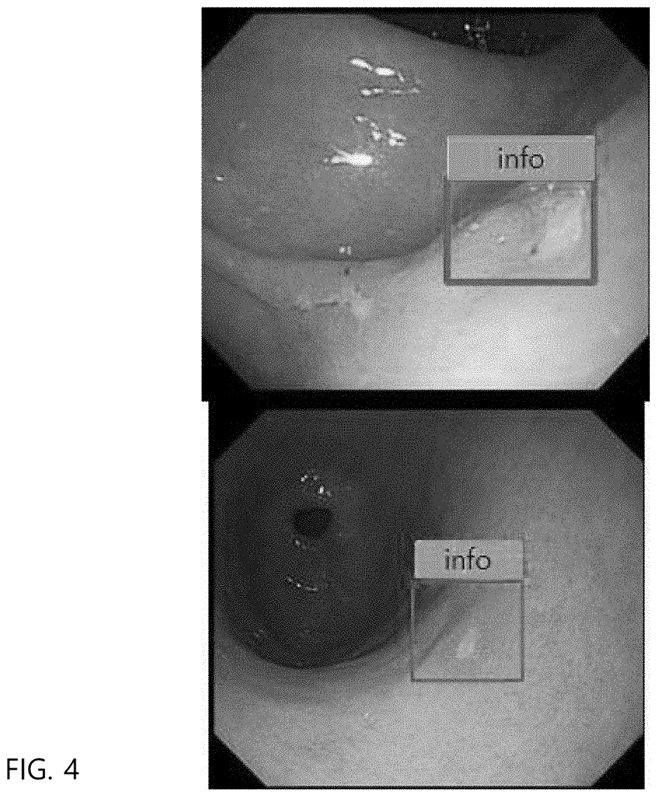

FIG. 4 shows views illustrating examples of an image in which a gastroscopic image and display information are displayed together according to an embodiment of the present invention.

The display information may include whether a finding suspected of being a lesion is present, the coordinates of the location of the finding suspected of being a lesion (the coordinates of a location within a current video frame), and the location of the finding suspected of being a lesion in a gastroscopic path.

The finding suspected of being a lesion is visualized to be visually distinguished from other parts in the video frame of the gastroscopic image, as shown in FIG. 4. In this case, as shown in FIG. 4, the corresponding finding may be marked with a visualization element such as a marker/box, or may be highlighted.

Furthermore, information about the location and the probability that the lesion in question is actually a lesion (the probability inferred by artificial intelligence) are included in the display information so that it can be visualized such that a user can intuitively understand the proximity or relevance of the finding suspected of being a lesion to the visualization element.

In the learning process of an artificial intelligence analysis algorithm for a gastroscopic image according to an embodiment of the present invention, training input data includes the following: A gastroscopic image used as the training input data includes an image, including a black background having a size varying depending on the resolution supported by an image acquisition device (a gastroscopic image acquisition module). In order to use only gastroscopic image information, endoscopic cropping is performed prior to learning. In a learning (training) stage, a location in a gastroscopic path may be learned along with information for the detection of a lesion (in the state of being included in label information), and, finally, the coordinate values where the lesion is located, the probability of being a lesion, and the result of the location in a path may be learned.

In an inference process for a real-time image after learning, the result value of the analysis is displayed on the user screen by using a visualization element that can be visually distinguished. In order to further reduce a user's risk of missing, when a risky finding is found, the user's attention may be called by using an alarm sound additionally. When the risk of mission is high due to the type of risky finding, the probability that the risky finding is a lesion, or the location of the risky finding in a blind spot in the field of view, different alarm sounds may be used in respective cases in order to further call the user's attention.

When training data is generated, the augmentation of data may be performed in order to resolve overfitting attributable to a specific bias (the color, brightness, resolution, or tilting of an endoscope device) of the data. The augmentation of data may be achieved through the rotation/tilting, movement, symmetry, and correction of the color/brightness/resolution of image data.

In addition, as an example of a method for preventing overfitting, there may be used various methods such as weight regulation, dropout addition, and network capacity adjustment (reduction).

In the embodiments of FIGS. 1 to 4, the real-time image acquisition module acquires a real-time endoscopic image from the endoscopic image diagnosis acquisition module/endoscopic equipment. The real-time image acquisition module transmits the real-time endoscopic image to the diagnosis assisting system. The diagnosis assisting system includes at least two artificial intelligence algorithms, and generates display information including diagnosis information by applying the at least two artificial intelligence algorithms to the real-time endoscopic image. The diagnosis assisting system transfers the display information to the user system, and the user system may overlay the display information on the real-time endoscopic image or display the real-time endoscopic image and the display information together.

The real-time endoscopic image may be divided into individual image frames. In this case, the endoscopic image frames may be received by the receiving interface.

The diagnosis assisting system (a computing system) includes the reception interface module, the processor, the transmission interface module, and the memory/storage. The processor includes sub-modules the functions of which are internally implemented by hardware or software. The processor may include a first sub-module configured to extract context-based diagnosis requirements, a second sub-module configured to select artificial intelligence analysis results to be displayed from among diagnosis results generated by applying artificial intelligence diagnosis algorithms to the endoscopic image frame, and a third sub-module configured to generate the display information to be displayed on the screen of the user system.

The plurality of artificial intelligence diagnosis algorithms may be stored in the memory or database (not shown) inside the diagnosis computing system, may be applied to the endoscopic image frame under the control of the processor, and may generate diagnosis results for the endoscopic image frame.

Although a case where the plurality of artificial intelligence diagnosis algorithms is stored in the memory or database (not shown) inside the diagnosis computing system and run under the control of the processor is described in the embodiments of FIGS. 1 to 4, the plurality of artificial intelligence diagnosis algorithms may be stored in memory or a database (not shown) outside the diagnosis computing system according to another embodiment of the present invention. When the plurality of artificial intelligence diagnosis algorithms is stored in the memory or database (not shown) outside the diagnosis computing system, the processor may control the memory or database (not shown) outside the diagnosis computing system via the transmission module so that the plurality of artificial intelligence diagnosis algorithms is applied to the endoscopic image frame and diagnosis results for the endoscopic image frame are generated. In this case, the generated diagnosis results may be transferred to the diagnosis computing system through the receiving interface, and the processor may generate the display information based on the diagnosis results.

The processor extracts diagnosis requirements for the endoscopic image frame by analyzing the endoscopic image frame, which is an image frame of a medical image. The processor selects a plurality of diagnosis application algorithms to be applied to the diagnosis of the endoscopic image frame from among the plurality of medical image diagnosis algorithms based on the diagnosis requirements, and the processor generates the display information including diagnosis results for the endoscopic image frame by applying the plurality of diagnosis application algorithms to the endoscopic image frame. This process is performed on each of the endoscopic image frames by the processor.

The processor may extract context-based diagnosis requirements corresponding to the characteristics of the endoscopic image frame by analyzing the endoscopic image frame. The processor may select a plurality of diagnosis application algorithms to be applied to the diagnosis of the endoscopic image frame based on the context-based diagnosis requirements.

The processor may select a combination of a plurality of diagnosis application algorithms based on the context-based diagnosis requirements. The processor may generate the display information including diagnosis results for the endoscopic image frame by applying the plurality of diagnosis application algorithms to the endoscopic image frame.

The combination of a plurality of diagnosis application algorithms may include a first diagnosis application algorithm configured to be preferentially recommended for the endoscopic image frame based on context-based diagnosis requirements, and a second diagnosis application algorithm configured to be recommended based on a supplemental diagnosis requirement derived from the context substrate diagnosis requirements based on a characteristic of the first diagnosis application algorithm.

The context-based diagnosis requirements may include one or more of a body part of the human body included in the endoscopic image frame, an organ of the human body, a relative location indicated by the endoscopic image frame in the organ of the human body, the probabilities of occurrence of lesions related to the endoscopic image frame, the levels of risk of the lesions related to the endoscopic image frame, the levels of difficulty of identification of the lesions related to the endoscopic image frame, and the types of target lesions. When an organ to which the endoscopic image frame is directed is specified, for example, when the endoscopic image frame is related to a colonoscopic image, information about whether the image displayed in the current image frame is the beginning, middle, or end of the colonoscopic image may be identified along with the relative location thereof in the colon (the inlet, middle, and end of the organ). In the case of a gastroscopic image, information about whether the image displayed in the current image frame is the beginning (e.g., the esophagus), middle (the inlet of the stomach), or end of the gastroscopic image may be identified along with the relative location thereof in a gastroscopic path.

Accordingly, the context-based diagnosis requirements may be extracted based on the types of lesions/diseases that are likely to occur at the identified location and region, the types of lesions/diseases that are likely to be overlooked by medical staff because they are difficult to identify with the naked eye, diagnosis information about lesions/diseases that are not easy to visually identify within the current image frame, and the types of lesions/diseases requiring attention due to their high risk/lethality during a diagnosis among the lesions/diseases that may occur at locations within the organ of the human body to which the current image frame is directed. In this case, the context-based diagnosis requirements may also include information about the types of target lesions/diseases that need to be first considered in relation to the current image frame based on the information described above.

The display information may include the endoscopic image frame, the diagnosis results selectively overlaid on the endoscopic image frame, information about the diagnosis application algorithms having generated the diagnosis results, and evaluation scores for the diagnosis application algorithms. The above-described process of calculating evaluation scores in the embodiments of FIGS. 1 and 2 may be used as the process of calculating the evaluation scores for the diagnosis application algorithms.

Although priorities may be allocated to the artificial intelligence diagnosis algorithm in descending order of evaluation scores in the application of diagnoses, there are some additional factors to be taken into consideration.

When a first-priority artificial intelligence algorithm detects a part of the lesions that are likely to occur in connection with the corresponding endoscopic image and a subsequent-priority artificial intelligence algorithm detects an item that is not detected by the first priority algorithm, both the diagnosis results of the first-priority artificial intelligence algorithm and the diagnosis results of the subsequent-priority artificial intelligence algorithm may be displayed together. Furthermore, there may be provided a menu that allows a user to select a final diagnosis application artificial intelligence algorithm based on the above-described criteria. In order to help the user to make a selection, the menu may be displayed together with the diagnosis results of the plurality of AI algorithms and a description of the reason for displaying the diagnosis results.

For example, it is assumed that lesions A1 and A2 are known as being the types of lesions that are most likely to occur within the current image frame and a lesion B is known as being less likely to occur than lesions A1 and A2 and being likely to be overlooked because it is difficult to visually identify. An artificial intelligence diagnosis algorithm X, which has obtained the highest evaluation score for the lesions A1 and A2, may obtain the highest overall evaluation score and be selected as the first diagnosis application algorithm that is preferentially recommended. Meanwhile, there may be a case where the first diagnosis application algorithm obtains the highest evaluation score for the lesions A1 and A2 but obtains an evaluation score less than a reference value for the lesion B. In this case, the lesion B for which the first diagnosis application algorithm exhibits the performance less than the reference value may be designated as a supplemental diagnosis requirement. An artificial intelligence diagnosis algorithm Y that obtains the highest evaluation score for the lesion B, which is a supplemental diagnosis requirement, may be selected as a second diagnosis application algorithm. A combination of the first and second diagnosis application algorithms may be selected such that the combination has high evaluation scores for the reliability and accuracy of the overall diagnostic information, the diagnostic information for a specific lesion/disease is prevented from being overlooked, and the diagnostic performance for a specific lesion/disease is prevented from being poor. Accordingly, logical conditions for the selection of a diagnosis application algorithm may be designed such that an artificial intelligence diagnosis algorithm exhibiting the best performance for the supplemental diagnosis requirement for which the first diagnosis application algorithm is weak, rather than the AI diagnosis algorithm exhibiting a high overall evaluation score, is selected as the second diagnosis application algorithm.

Although the case where the two diagnosis application algorithms are selected has been described as an example in the above embodiment, an embodiment in which three or more diagnosis application algorithms are selected and applied may also be implemented according to the description given herein in the case where the combination of the three or more diagnosis application algorithms exhibits better performance according to the evaluation scores.

The embodiments of FIGS. 1 to 4 are embodiments in which the diagnosis results obtained by the application of the artificial intelligence diagnosis algorithms having high internal evaluation scores are presented and then a user may select the diagnosis results obtained by the application of artificial intelligence diagnosis algorithms having higher evaluation scores. In the embodiment of FIGS. 1 to 4, there is disclosed a configuration conceived for the purpose of rapidly displaying diagnosis results for a real-time endoscopic image. Accordingly, in the embodiment of FIGS. 1 to 4, a combination of artificial intelligence diagnosis algorithms to be displayed for the current image frame is preferentially selected based on context-based diagnosis requirements, the diagnosis results of this combination are generated as display information, and the display information together with the image frame is provided to a user.

In this case, the types of lesions/diseases that are likely to occur in the current image frame, the types of lesions/diseases that are likely to occur in the current image frame and are also likely to be overlooked by medical staff because they are difficult to visually identify, and the types of lesions/diseases requiring attention during diagnosis due to their high risk/lethality among the lesions that may occur in the current image frame may be included in the context-based diagnosis requirements. Furthermore, the types of target lesions/diseases that should not be overlooked in the current image frame based on the types and characteristics of lesions/diseases, and the priorities of the types of target lesions/diseases may be included in the context-based diagnosis requirements.

When the diagnosis results and display information of the present invention are used in a hospital, they are displayed by the user system having a user interface capable of displaying auxiliary artificial intelligence diagnosis results after endoscopic data has been received and analyzed, and then the diagnosis results may be verified, the diagnosis results may be replaced, or the acceptance or rejection of the diagnosis results may be determined based on user input.

The processor may store the display information in the database with the display information associated with the endoscopic image frame. In this case, the database may be a database inside the diagnosis computing system, and may be stored as medical records for a patient in the future.

The processor may generate external storage data in which the display information and the endoscopic image frame are associated with each other, and may transmit the external storage data to an external database via the transmission module so that the external storage data can be stored in the external database. In this case, the external database may be a PACS database or a database implemented based on a cloud.

In this case, the plurality of medical image diagnosis algorithms are artificial intelligence algorithms each using an artificial neural network, and the processor may generate evaluation scores based on the respective diagnosis requirements/context-based diagnosis requirements as descriptive information for the plurality of respective medical image diagnosis algorithms.

The diagnosis assisting system of the present invention may internally include at least two artificial intelligence diagnosis algorithms. Endoscopic image data is transferred from three or more pieces of endoscopy equipment to the diagnosis assisting system. The diagnosis assisting system generates diagnosis results by applying the at least two artificial intelligence diagnosis algorithms to each frame of the endoscopic image data. The diagnosis assisting system generates display information by associating the diagnosis results with the frame of the endoscopic image data. In this case, the display information may be generated to include the identification information of a hospital (hospital A) in which the endoscopic image data is generated. Furthermore, the display information may be generated to include the identification information (endoscope 1, endoscope 2, or endoscope 3) given to each piece of endoscope equipment of each hospital.

The diagnosis assisting system of the present invention transmits the generated display information to a cloud-based database, and the endoscopic image data and the display information are stored in the cloud-based database in the state in which the endoscopy equipment, in which the endoscopic image data was generated, and the hospital, in which the endoscopic image data was generated, are identified. The display information may be generated by associating diagnosis information with each frame of the endoscopic image data and then stored. The diagnosis information generated for each frame of the endoscopic image data may be automatically generated based on evaluation scores and context-based diagnosis requirements, as described in the embodiments of FIGS. 1 to 4.

When the present invention is applied in a cloud environment, endoscopic image data and diagnosis results may be received by a user system on a hospital side using equipment connected over a wireless communication network, and auxiliary artificial intelligence diagnosis results may be displayed on the user system.

The display information stored in the cloud database may be provided to a hospital designated by a patient, and the patient may receive his or her endoscopic image data and diagnosis information at a hospital that is convenient to access and also receive a doctor's interpretation of diagnosis results and a follow-up diagnosis at the hospital.

Diagnosis results are generated using results, to which artificial intelligence algorithms are applied, by the diagnosis computing terminal of the medical staff. In this case, the comments of the medical staff may be added during the process of generating diagnosis results.

In the medical image diagnosis assisting system according to the present invention, the I-scores, i.e., evaluation scores, are transferred from the computing system to the diagnosis computing terminal of the medical staff. Final diagnosis texts may be generated by incorporating the I-scores, i.e., evaluation scores, into the generation of the diagnosis results. According to an embodiment of the present invention, the computing system may generate diagnosis texts together with the I-scores, i.e., evaluation scores, and transfer them to the computing system of the medical staff. In this case, the diagnosis texts generated by the computing system may be written using the diagnosis results based on diagnosis application algorithms having higher I-scores, i.e., higher evaluation scores.

The computing system may provide a user interface configured to allow recommended diagnosis results to be selected using I-scores, i.e., internally calculated evaluation scores, and to allow a radiologist to evaluate/check diagnostic confidence in corresponding recommended diagnoses (e.g., recommended diagnosis algorithms consistent with the diagnosis results of the radiologist) because the evaluation scores are also displayed. The processor of the computing system may select the first and second diagnosis results from among the plurality of diagnosis results as recommended diagnosis results based on the evaluation scores. The processor may generate display information, including the evaluation score for the first diagnosis algorithm, the first diagnosis result, the evaluation score for the second diagnosis algorithm, and the second diagnosis result.

The computing system may generate an evaluation score based on the confidence score of a corresponding diagnosis algorithm, the accuracy score of the diagnosis algorithm, and the evaluation confidence score of a radiologist who provides feedback. The processor may generate the confidence score of each of the plurality of medical image diagnosis algorithms, the accuracy score of the medical image diagnosis algorithm, and the evaluation confidence score of the medical image diagnosis algorithm by the user as sub-evaluation items based on a corresponding one of the plurality of diagnosis results and feedback on the diagnosis result, and may generate an evaluation score based on the sub-evaluation items.

For example, the criteria for the generation of the evaluation score may be implemented as follows:

I-score=a×(the confidence score of an artificial intelligence algorithm)+b×(the accuracy score of the artificial intelligence algorithm)+c×(the evaluation confidence score of the artificial intelligence algorithm by a radiologist) (1)

The confidence score of the algorithm may be given to the algorithm by the radiologist. In other words, when it is determined that the first diagnosis result is more accurate than the second diagnosis result, a higher confidence score may be given to the first diagnosis result.

The accuracy score of the algorithm may be determined based on the extent to which the radiologist accepts the diagnosis result of the algorithm without a separate score giving process. For example, in the case where when the first diagnosis result presents ten suspected lesion locations, the radiologist approves nine suspected lesion locations, the accuracy score may be given as 90/100.

Another embodiment in which the accuracy score of the algorithm is given may be a case where an accurate result is revealed through a biopsy or the like. In this case, the accuracy of the diagnosis result of the diagnosis algorithm may be revealed in comparison with the accurate result obtained through the biopsy. When the user inputs the accurate result, obtained through the biopsy, to the computing system, the computing system may calculate the accuracy score of the diagnosis algorithm by comparing the diagnosis result with the accurate result obtained through the biopsy (a reference).

The evaluation confidence score of the radiologist may be provided as a confidence score for the evaluation of the radiologist. In other words, when the radiologist is an expert having a loner experience in a corresponding clinical field, a higher evaluation confidence score may be given accordingly. The evaluation confidence score may be calculated by taking into consideration the years of experience of the radiologist, the specialty of the radiologist, whether or not the radiologist is a medical specialist, and the experience in the corresponding clinical field.

The computing system may update evaluation score calculation criteria according to a predetermined internal schedule while continuously learning the evaluation score calculation criteria by means of an internal artificial intelligence algorithm. The processor may assign weights to the confidence scores of the plurality of respective medical image diagnosis algorithms, the accuracy scores of the plurality of respective medical image diagnosis algorithms, and the evaluation confidence scores of the plurality of respective medical image diagnosis algorithms by the user, which are sub-evaluation items, and may update the weights of the sub-evaluation items so that the weights of the sub-evaluation items can be adjusted according to a target requirement based on the plurality of diagnosis results and feedback on the plurality of diagnosis results by the user.

An example of the target requirement may be a case where adjustment is performed such that there is a correlation between the confidence of the user in the algorithms and the accuracy of the algorithms. For example, first and second diagnosis algorithms having the same accuracy score may have different confidence scores that are given by a radiologist. In this case, when confidence scores are different from each other while exhibiting a certain tendency after the removal of the general errors of the evaluation of the radiologist, it can be recognized that the confidence of the radiologist in the first diagnosis algorithm is different from the confidence of the radiologist in the second diagnosis algorithm. For example, in the case where the first and second diagnosis algorithms generate accurate diagnosis results at nine of a total of ten suspected lesion locations, resulting in an accuracy score of 90/100 but only the first diagnosis algorithm accurately identifies a severe lesion and the second diagnosis algorithm does not identify the lesion, the confidence of the radiologist in the first diagnosis algorithm may be different from the confidence of the radiologist in the second diagnosis algorithm. A means for adjusting the correlation between the accuracy and the confidence may be a means for adjusting the weights of the respective sub-evaluation items or subdividing criteria for the selection of target lesions related to the determination of accuracy. In this case, there may be used a method that classifies lesions according to criteria such as the hardness/severity of an identified lesion, the position of the lesion from the center of a medical image, and the difficulty of identifying the lesion (the difficulty is high in a region where bones, organs, and blood vessels are mixed in a complicated form) and assigns different weights to the diagnosis accuracies of lesions in respective regions.

The computing system may include a function of automatically allocating a plurality of artificial intelligence algorithms that are applicable depending on an image. To determine a plurality of artificial intelligence algorithms applicable to an image, the computing system 100 may classify one examination or at least one image by means of a separate image classification artificial intelligence algorithm inside a recommendation diagnosis system, and may then apply a plurality of artificial intelligence algorithms.

In an embodiment of the present invention, the plurality of medical image diagnosis algorithms may be medical image diagnosis algorithms using artificial neural networks. In this case, the evaluation score and the sub-evaluation items may be generated as descriptive information for each diagnosis algorithm, and the computing system 100 may feed the evaluation score and the sub-evaluation items back to the creator of the diagnosis algorithm so that the information can be used to improve the diagnosis algorithm. In this case, when each of the artificial neural networks is an artificial neural network using a relevance score and a confidence level, which is being studied recently, a statistical analysis is performed with the evaluation score and the sub-evaluation items associated with the relevance score or confidence level of the artificial neural network, and thus the evaluation score and the sub-evaluation items may affect the improvement of the diagnosis algorithm.

This embodiment of the present invention is designed to provide advantages obtainable by the present invention while minimizing the deformation of the medical image diagnosis sequence of the related art as much as possible.

In another embodiment of the present invention, the computing system may perform the process of generating a plurality of diagnosis results by selecting a plurality of diagnosis application algorithms and then applying the plurality of diagnosis application algorithms to a medical image by itself. In this case, the computing system may transfer not only information about the selected diagnosis application algorithms but also the plurality of diagnosis results based on the diagnosis application algorithms to the diagnosis computing terminal of the medical staff, and the results obtained by applying artificial intelligence algorithms (the diagnosis application algorithms) to the medical image may be displayed on the diagnosis computing terminal of the medical staff.

In this case, an embodiment of the present invention may provide advantages obtainable by present invention even when the computing power of the diagnosis computing terminal of the medical staff is not high, e.g., the diagnosis computing terminal of the medical staff is a mobile device or an old-fashioned computing system. In this case, in an embodiment of the present invention, an agent that applies the artificial intelligence algorithms to the medical image is the computing system, the computing system functions as a type of server, and the diagnosis computing terminal of the medical staff may operate based on a thin-client concept. In this case, in an embodiment of the present invention, the feedback indicators input for the plurality of diagnosis results or the plurality of diagnosis application algorithms via the diagnosis computing terminal of the medical staff by the medical staff may be fed back to the computing system. The feedback indicators may be stored in the memory or database inside the computing system in association with the evaluation targets, i.e., the plurality of diagnosis results or the plurality of diagnosis application algorithms.

As described above, in an embodiment of the present invention, the step of applying the selected algorithms may be performed in the diagnosis system of the clinician, and a plurality of diagnosis results may be transferred to the computing system. In another embodiment of the present invention, the overall step of applying the selected algorithms may be performed within the computing system and then the results of the application may be displayed on the diagnosis system of the clinician.

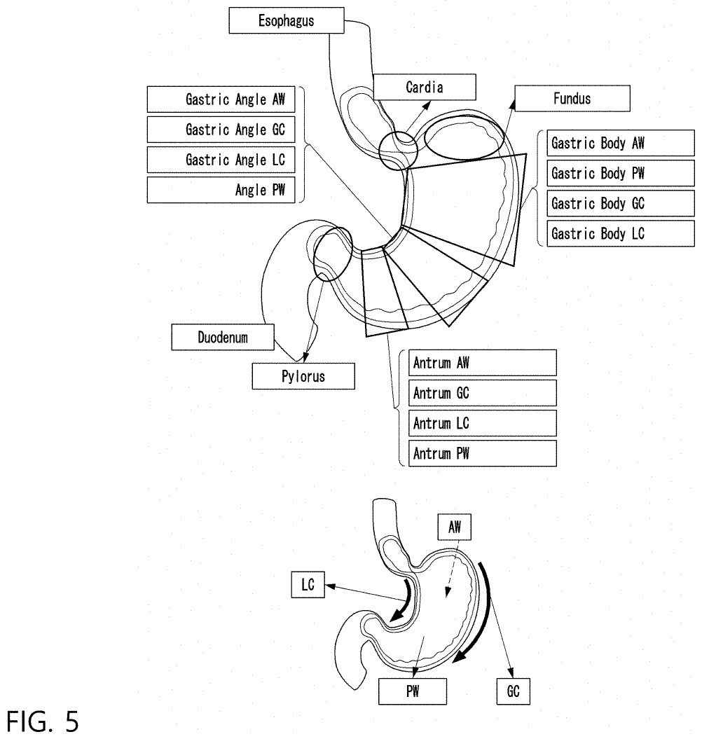

FIG. 5 is an example of a process of classifying an anatomical position in the stomach during gastroscopy performed by a gastroscopic image diagnosis supporting system according to an embodiment of the present invention.

According to one embodiment of the present invention, anatomical position classes inside the stomach may be divided into the esophagus, the fundus, the cardia, the body, the angle, the antrum, the pylorus, and the duodenum. In this case, each of the body, the angle, and the antrum may be divided into an anterior wall (AW), a posterior wall (PW), a Greater Curvature (GC), and a Lesser Curvature (LC). As a result, a total of 17 classes may be obtained.

The anatomical position classes in the stomach according to the present invention are not limited to the embodiment shown in FIG. 5, and it will be apparent to those of ordinary skill in the art that classes such as those disclosed in Korean Patent No. 10-2255311 may be used.

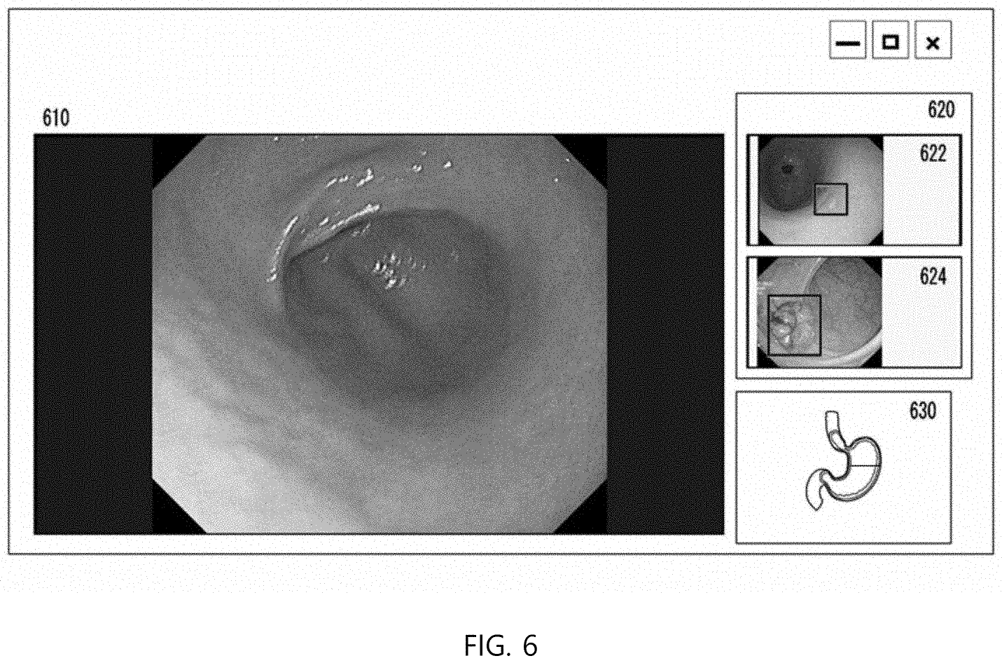

FIG. 6 is an example of display information and/or a user interface that are provided together with a gastroscopic image by the gastroscopic image diagnosis supporting system according to the embodiment of the present invention.

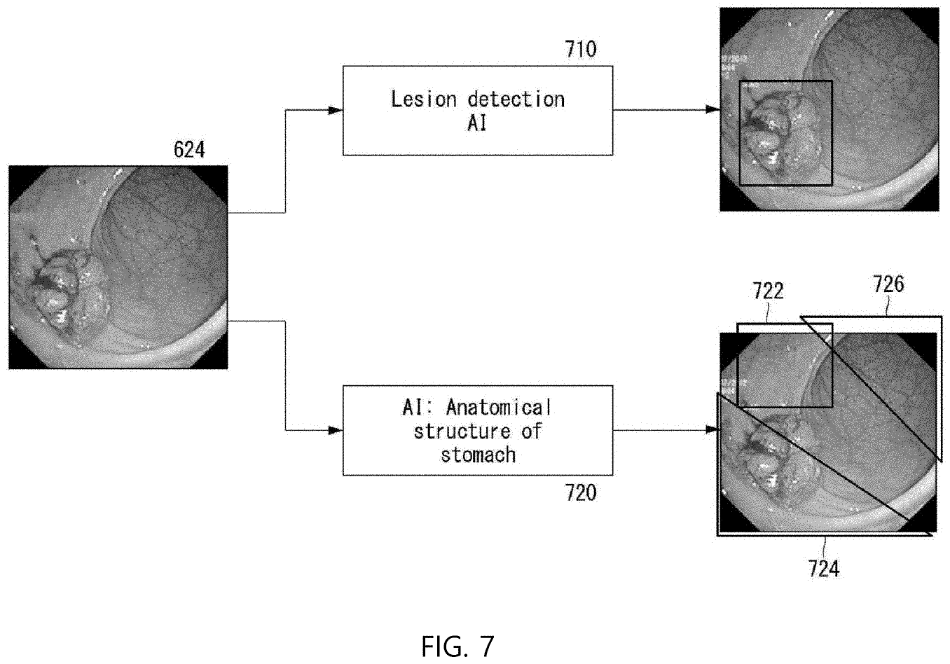

FIG. 7 is an example of a process of detecting a lesion in a gastroscopic image and classifying an anatomical position that is performed by the gastroscopic image diagnosis supporting system according to the embodiment of the present invention.

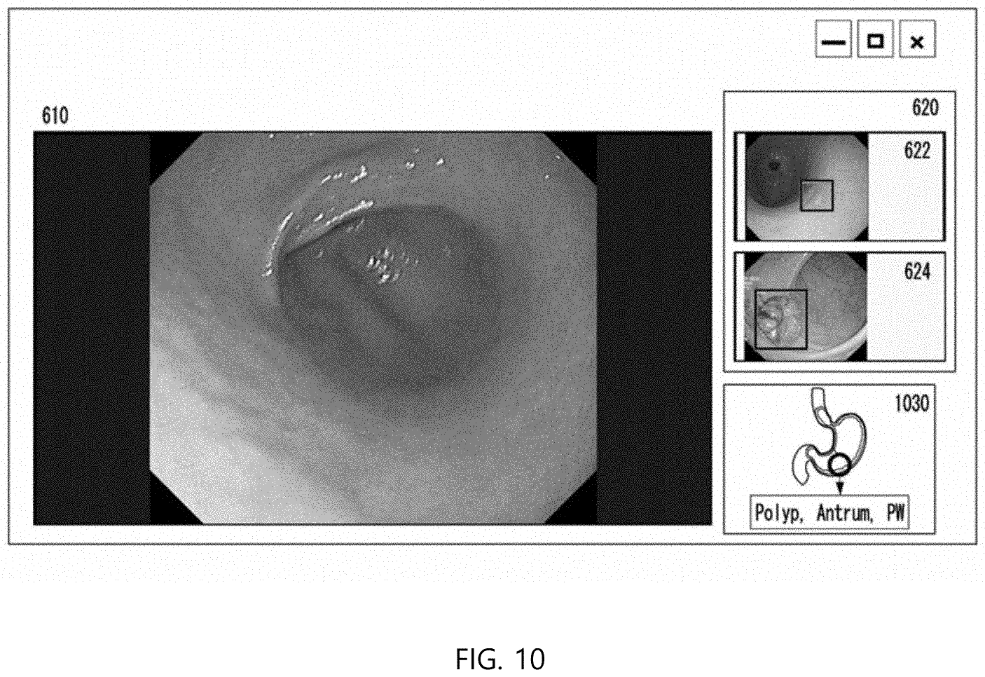

FIG. 10 is an example of display information and/or a user interface that are provided together with a gastroscopic image by the gastroscopic image diagnosis supporting system according to the embodiment of the present invention.

A real-time gastroscopic image may be displayed in the first window 610 of FIG. 6. By a cropping process for each frame of a gastroscopic image, the black portions of the edges of the gastroscopic image are removed, and only a valid portion is transmitted to artificial intelligence models 710 and 720. Although the two artificial intelligence models 710 and 720 are shown in the present embodiment of the present invention, another artificial intelligence model for classifying the type of detected lesion may be provided. In this case, according to an embodiment of the present invention, the detection of a lesion and the classification of the type of detected lesion may be simultaneously performed by the lesion detection artificial intelligence model 710. Whether a lesion has been detected may be marked with a marker 712. When the type of detected lesion is classified, whether the lesion is a polyp, gastric cancer, or gastric ulcer may be determined and marked with one of the markers 622 and 624 having different visualization elements. In this case, the different visualization elements may be distinguished by different colors, patterns, the types of the outlines of markers (dotted lines or solid lines), etc. according to the type of lesion.

In the second window 620 of FIG. 6, finding information (a captured frame) about the detected lesion may be displayed. When one or more polyps, gastric ulcers, and/or gastric cancers are found through the lesion detection artificial intelligence model 710, they may be accumulated and displayed in a list format on the second window 620. The lesions and/or finding information about the lesions displayed in the second window 620 may be displayed in the separate areas of the second window 620 by taking into consideration the types, degrees of importance, sizes, and/or number of lesions, and/or the number of a specific type of lesions.

In the third window 630 of FIG. 6, a gastrointestinal anatomical position map is displayed, and an anatomical position (part) in the stomach corresponding to an image currently being displayed in the first window 610 may be classified by the gastric anatomical structure artificial intelligence model 720. The classified anatomical position currently being examined or one or more anatomical positions/paths examined so far may be displayed by the third window 630. In another embodiment 1030 of the third window, one or more anatomical positions/paths examined so far may be displayed, an anatomical position currently being examined may be highlighted and displayed, and whether a lesion has been detected at a corresponding anatomical position, the type of detected lesion, and a brief description (the class) of the anatomical position may be displayed. The third window 630 or the other embodiment 1030 of the third window may be visualized in the process of displaying an endoscopic image under examination in step S940, and may also be visualized in the process in which a user refers to an endoscopic image for verification, confirmation, or decision-making after the termination or temporary termination of examination in step S942, S944, or S950. In the display process S942, S944, or S950 after the termination or temporary termination of examination, there may be added a navigation function in which the anatomical position of an endoscopic image currently being displayed in the first window 610, whether a lesion has been detected, the type of detected lesion, and the path to the anatomical position are displayed such that a user can rapidly reach the anatomical position where a major lesion is present and a video frame corresponding to the position.

The gastric anatomical structure artificial intelligence model 720 may search for a class 722, 724, or 726 corresponding to a current image by identifying the classes 722, 724, and 726 corresponding to respective anatomical positions in the image, and may determine the anatomical position of the current image. In this case, the gastric anatomical structure artificial intelligence model 720 may determine an anatomical position corresponding to a class having the highest degree of fit or the largest weight to be the anatomical position of a current image by taking into consideration the weights of the classes 722, 724, and 726 identified in the current image.

In FIG. 6, when the anatomical position of a current gastroscopic image is recognized, an anatomical structure in the stomach corresponding to a path passed through so far is displayed in the third window 630. The anatomical structure in the stomach corresponding to the path passed through may be visualized to be distinguished from an anatomical structure that has not yet been examined by using one or more different visualization elements (a color, a pattern, and/or a highlight). In the present invention, a user's reading, diagnosis, and/or decision-making may be supported such that distinctive visualization based on whether an anatomical structure has been examined prompts a user to check information about a blind spot that has not yet been passed through in current examination once more in real time during examination, thereby allowing the user to examine one or more lesions that have not been checked or have been missed. Through this configuration of the present invention, the overall lesion detection rate may be improved.

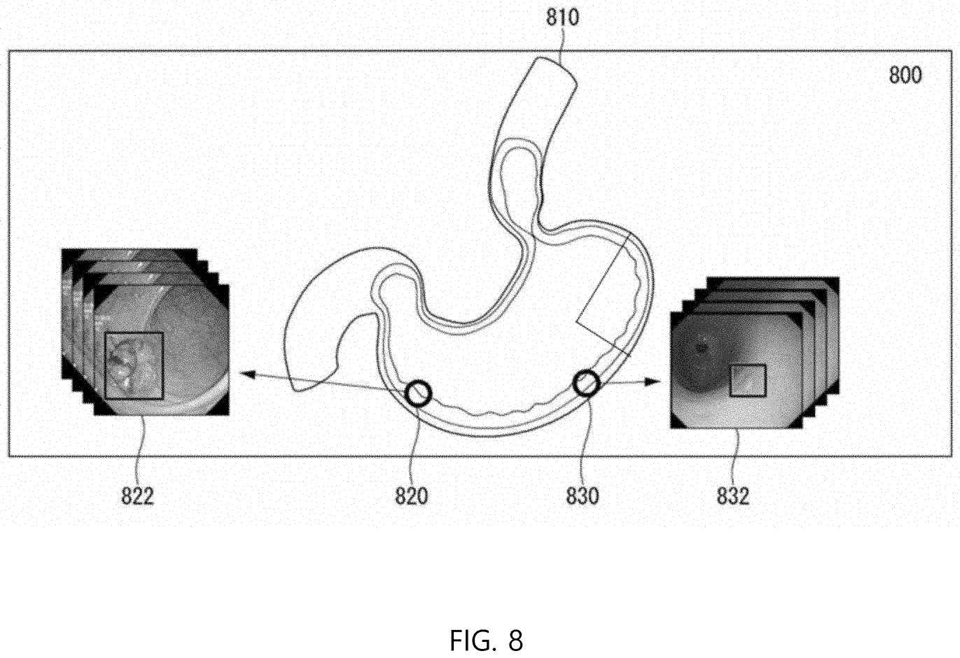

FIG. 8 shows an example of a user interface provided by the gastroscopic image diagnosis supporting system according to the embodiment of the present invention, and is a diagram showing a user interface for lesion findings displayed together with a gastrointestinal anatomical position map during or after examination and a process of calling findings.

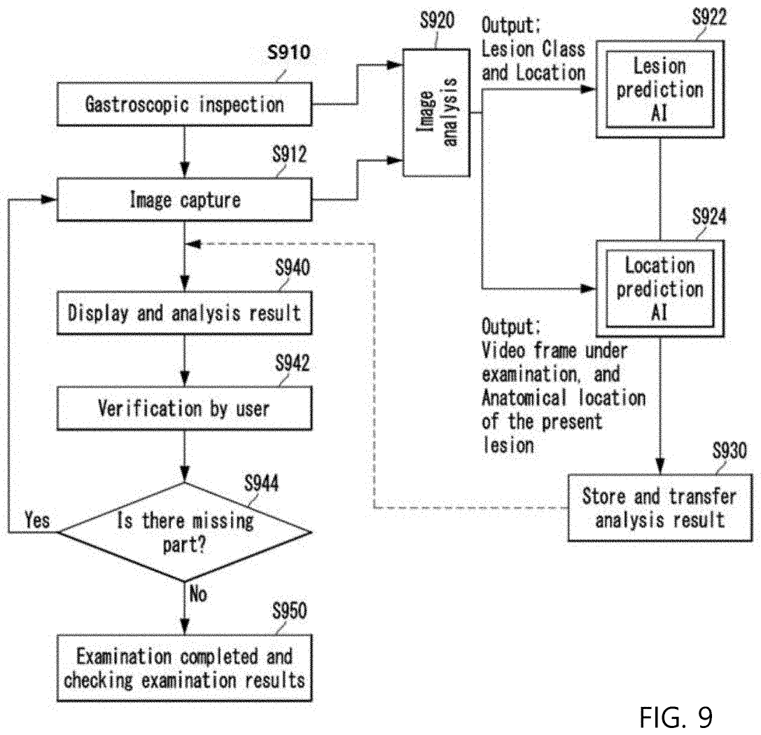

FIG. 9 is an operational flowchart showing the workflow of a method performed by the gastroscopic image diagnosis supporting system according to the embodiment of the present invention.

Referring to FIGS. 5 to 10, the system according to the embodiment of the present invention performs gastroscopy in step S910. According to a user request or a predetermined sequence, the image capture of a video frame in a gastroscopic image may be performed in step S912.

Gastroscopy is an examination method that identifies digestive diseases appearing in the esophagus, stomach, duodenum, etc. by inserting a gastroscope into the human body. This plays an important role in the early detection and treatment of diseases such as gastric ulcer and gastric cancer.

In particular, detailed imaging, observation, and recording are important for the early detection of gastric cancer, and it is most important to ensure that no lesions are missed. However, there are cases where up to 25% of lesions are missed due to imaging problems, blind spots, human error, and doctors' fatigue caused by continuous and repetitive procedures, which negatively affects medical results.

In addition, when adenoma, gastric cancer, or another abnormal finding is found through biopsy due to a microscopic lesion, endoscopy is often required again. However, there are many cases in which a small lesion that was suspected during first endoscopic examination and biopsied is not found during second examination. Accordingly, it is necessary to accurately record the position and shape of the lesion in detail. This is a considerable burden for medical professions. Even when it is recorded in detail, subjective determination is involved in this case, and also the shape of a biological tissue may be subtly changed, so that there is still a problem in that it is difficult to accurately find the lesion of a corresponding finding during a reexamination process.

This problem is present not only during reexamination but also during follow-up examination such as periodic health examination. In particular, in the case of a patient with multiple ulcers and polyps, there are many cases where it is not easy to search for a lesion corresponding to a past finding in a current image, so that there has emerged a need for an effective user interface that can easily assist in finding the position of a lesion corresponding to the finding, in accessing a record of the past finding for the lesion, and in searching for the past finding. The present invention may provide a reading support function through an intuitive and effective user interface.

In addition, when a picture of an endoscopic finding is left, it is also important to capture/take a picture so that the characteristic finding of a lesion can be represented well in the picture because the position or overall characteristic of the lesion cannot be determined when the picture is captured/taken excessively close. This is an essential part for reexamination when adenoma, gastric cancer, or another abnormal finding is found after a biopsy on a microscopic lesion. The user interface of a feature that helps to keep track of what has been found is very important. A user interface having a function that automatically helps to track an anatomical position at which a lesion (a polyp) was found is significantly important because there are cases where the lesion (the polyp) cannot be found again during reexamination.

In gastroscopy, not only the presence or absence of a lesion but also the anatomical position of the lesion are considered to be important information. In the present invention, there is provided the intuitive user interface that allows a user to easily search for or access existing finding information during subsequent review, reexamination, or follow-up examination of examination results by taking into consideration the anatomical position information of the lesion.

The present invention improves the lesion detection rate by reducing blind spots, which are missing parts during examination, by providing the user interface that automatically finds lesions during gastroscopy and displays a path passed through. Whether a lesion was detected at each position may be visualized by showing the sequence of a path passed through and displaying statistical information on how many lesions were detected at individual anatomical positions and images of found results after the completion of examination.

Accordingly, the present invention may help a user to easily take follow-up measures by using examination results, to easily make a follow-up decision, or to easily search for or access the finding information of previous examination in the course of follow-up examination or reexamination.

According to the results of a previous study (D. A. Corley et al., “Adenoma Detection Rate and Risk of Colorectal Cancer and Death”), it is known that in particular, a 1.0% increase in adenoma detection rate correlates with a 3.0% decrease in cancer incidence. Accordingly, an object of the present invention is to increase lesion detection rate and to lower the incidence of gastric cancer by filtering out gastric cancer risk factors in an early stage. Additional objects of the present invention are to contribute to reducing the causes of gastric cancer by enabling a doctor to find and treat more lesions than before, and to contribute to reducing the frequency of examinations.

Through the present invention, work efficiency and diagnosis accuracy may be increased by training an artificial intelligence algorithm with polyps, ulcers, cancers, various gastrointestinal diseases, etc. that may be missed during gastroscopy and applying it to an artificial intelligence diagnosis supporting system. In an embodiment of the present invention, at least two types of artificial intelligence may be used. Artificial intelligence that analyzes polyps, ulcers, cancer, etc., and artificial intelligence that analyzes the position information of the current gastric wall may be used. Classes are divided according to the anatomical position in the stomach, and there is provided a user interface that adds a mark indicating that examination has been completed to a part corresponding to a class for which examination has been completed to visualize an unexamined region (a blind spot) so that it can be easily identified. The adenoma detection rate may be improved by the user interface. In addition, there may be included a function that facilitates position tracking by indicating the gastrointestinal anatomical position of a lesion found by artificial intelligence and supports the easy tracking of a lesion of previous examination in reexamination or follow-up examination after the termination of examination. As a result, the effect of reducing the incidence of gastric cancer is expected via the reading support function.

A gastroscopic image diagnosis supporting system according to an embodiment of the present invention includes a computing system, and the computing system includes a reception interface, memory or a database, and a processor. The reception interface receives a medical image, and the memory or database stores one or more medical image analysis models 710 and 720 each having the function of analyzing a medical image (a gastroscopic image).

The processor analyzes the video frame of a gastroscopic image by using the first medical image analysis model 720 in step S920, and classifies the gastrointestinal anatomical position of the video frame by identifying a part corresponding to a preset gastrointestinal anatomical position class in the video frame in step S924.

When a user captures and stores a video frame as finding information about a gastrointestinal lesion in step S930, the processor stores the gastrointestinal anatomical position information of the video frame as the index information of the finding information together with the finding information. When the user (the examiner) captures a video frame for a region suspected of having a polyp, ulcer, or gastrointestinal disease within the video frame of a real-time gastroscopic image and stores a picture as finding information about a lesion (or a suspected region), the processor may store the position of the lesion or suspected region (classified by an artificial intelligence model) as an index for future retrieval together with the finding information.

The processor may provide display information including a gastrointestinal anatomical position map. The processor may visualize and display a gastrointestinal anatomical position by using the position map.

While the video frame is displayed to the user through a user display, the processor may display the gastrointestinal anatomical position of the video frame on a gastrointestinal anatomical position map and provide the gastrointestinal anatomical position map together with the video frame to the user.

When the user requests the checking of finding information after endoscopy, the processor may display the gastrointestinal anatomical position of the finding information on a gastrointestinal anatomical position map and provide the gastrointestinal anatomical position map to the user.

The processor may analyze the video frame of the gastroscopic image by using the second medical image analysis model 710 in step S920, and may detect whether a region suspected of being a lesion is present in the video frame in step S922.

An analysis result may be provided to the user through the user display by using the display information 610, 620, 630, and 800 in step S940.

The user may check the analysis result in step S942, and may check a detected lesion, a classification result for the type of detected lesion, and the position of the lesion.

In a user checking process, it is checked whether there is a missing part based on the information displayed through a map 810 in step S944.

When there is no missing part, examination is terminated, and a process of checking the examination result by a medical professional is performed in step S950. In this case, a decision is made on follow-up measures, and examples of the follow-up measures include the biopsy of a detected lesion, reexamination, follow-up examination (follow-up examination after a specific period of time or periodical follow-up examination), and the observation of a patient. When there is a missing part, examination is resumed, and an additional image capture may be performed as needed in step S912.

The processor may classify the type of lesion by comparing a region suspected of being a lesion against lesion classes by using a third medical image analysis model in step S922.

The processor may generate gastroscopy entry and exit path information based on information about the gastrointestinal anatomical position of the video frame and the sequential position at which the video frame is acquired.

The processor may provide the information to the user by displaying the gastroscopy entry and exit path information on a gastrointestinal anatomical position map through the user display.

In the checking process S942, S944, or S950 after the termination or temporary termination of the examination, the user may rapidly search for or rapidly access the finding information by using the position information stored together with the finding information. In addition, the user may rapidly search for a video frame, from which finding information is captured, among all video frames or access the corresponding video frame by using the entry and exit path information of gastroscopy and information about the sequential position at which the video frame is acquired. Although not shown in the drawings, a navigation menu for all video frames may be provided in relation to positions corresponding to the video frames, path information, and the sequence in which the video frames are acquired.

The processor may display a user interface for accessing the finding information of previous gastroscopy on a first gastrointestinal anatomical position map at the gastrointestinal anatomical position of the gastroscopic finding of the previous gastroscopy, and may provide the first gastrointestinal anatomical position map to the user. According to an embodiment of the present invention, the display information 800 shown in FIG. 8 may be provided for the previous gastroscopy. The display information 800 of FIG. 8 may be displayed to the user through a window separate from the first to the third windows 610, 620, and 630 of FIG. 6. In this case, the first gastrointestinal anatomical position map may correspond to the map 810, and the user may access the finding information of the previous gastroscopy through the user interfaces 820 and 830 (see 822 and 832).

The finding information (a captured image, whether a lesion has been detected, the type of detected lesion, or the like) of previous examination may be displayed on a first map, and the path of current examination may be displayed on a second map.

The processor may display the path of current gastroscopy and the gastrointestinal anatomical position of a current video frame on the second gastrointestinal anatomical position map, and may provide the second gastrointestinal anatomical position map to the user. According to an embodiment of the present invention, the display information 800 shown in FIG. 8 may be provided for current examination. In this case, the second gastrointestinal anatomical position map may correspond to the map 810, and the path of current gastroscopy and the gastrointestinal anatomical position of a current video frame may be displayed on the map 810. During or after examination, visualization may be performed on a result page such that the area where gastroscopy was not performed and the area where the gastroscopy was performed can be distinguished from each other. In this case, various visualization elements such as colors, patterns, markers, and highlights may be used as distinctive visualization elements. The distinction between the structure (part) in which gastroscopy was performed and the structure (part) in which gastroscopy was not performed may be provided even during examination and in the process S942, S944, and S950 in which the user checks or makes a decision using the examination result after the termination of examination or the temporary termination of examination.

Furthermore, display may be made such that the user can view at a glance the anatomical structure (part) from which a medical professional captured an image. A menu is provided to list the results of lesions (a gastric polyp, gastric ulcer, and/or gastric cancer) that were detected in a structure for a patient, and the user may view the results in detail by selecting individual menu options. Statistical information about the types of lesions detected in individual parts of the stomach may also be provided.

A gastroscopic image diagnosis supporting system according to another embodiment the present invention includes a computing system, and the computing system includes a reception interface, memory or a database, and a processor.

The processor analyzes the video frame of a gastroscopic image using the first medical image analysis model 720 in step S920, and classifies the gastrointestinal anatomical position of the video frame by identifying a part corresponding to a preset gastrointestinal anatomical position class in the video frame in step S924.

The processor displays whether a lesion was found at a position corresponding to each anatomical position class and statistical information about lesions found on a gastrointestinal anatomical position map at a position corresponding to each anatomical position class and provides the gastrointestinal anatomical position map to the user through the user display in step S930.

The processor may display whether a lesion was found at a position corresponding to each anatomical position class in the video frame of previous gastroscopy and statistical information about one or more lesions, found at the position corresponding to each anatomical position class, on the first gastrointestinal anatomical position map by using the analysis result of the video frame of the previous endoscopy. In this case, the detection and/or classification of the displayed lesions may be automatically performed by an artificial intelligence model, or may be determined by the user's input after the user's determination.

The lesion statistics of the previous examination and a position map (the first map) may be displayed separately, and the path of current examination may be displayed as a separate position map (the second map).

The processor may display the path of current endoscopic examination and the gastrointestinal anatomical position of a current video frame on the second gastrointestinal anatomical position map by using the analysis result of the video frame of the current endoscopic examination.

The processor may display whether a missing anatomical position class is present in the video frame of previous endoscopy and the anatomical position of the missing anatomical position class on the first gastrointestinal anatomical position map by using the analysis result of the video frame of the previous endoscopy. A blind spot corresponding to the anatomical position missed in the previous examination may be displayed on the first map.

A gastroscopic image diagnosis supporting system according to another embodiment of the present invention includes a computing system, and the computing system includes a reception interface, memory or a database, and a processor.

The processor analyzes the video frame of a gastroscopic image by using the first medical image analysis model 720 in step S920, and classifies the gastrointestinal anatomical position of the video frame by identifying a part corresponding to a preset gastrointestinal anatomical position class in the video frame in step S924.

The processor generates gastroscopy entry and exit path information based on information about the gastrointestinal anatomical position of the video frame and the sequential position at which the video frame is acquired.

The processor stores the gastrointestinal anatomical position information of the video frames and the entry and exit path information of the gastroscopy together with the video frames as the index information of the video frames.

The user interface may be provided to enable rapid search for lesions after the termination or temporary termination of examination along with the sequence information. The user interface may provide a function of providing support that the diagnosis sequence for individual parts in the entry of gastroscopy and the image display sequence for individual parts in the exit of gastroscopy, so that a part suspected of being a lesion can be rapidly searched for in the image frame data set of the overall gastroscopy after the determination of the examination.

The processor may provide a gastrointestinal anatomical position map.

The processor may display a user interface for accessing a video frame on the gastrointestinal anatomical position map at the gastrointestinal anatomical position of the video frame, and may provide the gastrointestinal anatomical position map to the user. The user may access the video frame of a corresponding position through the user interface on the map after the termination or temporary termination of examination.

The processor may display whether a video frame is present at a position corresponding to each anatomical position class, whether a missing anatomical position class missed is present in the video frame of gastroscopy, and an anatomical position on the gastrointestinal anatomical position map, and may provide the gastrointestinal anatomical position map to the user. The user may check a blind spot by referring to an examined area (the area where a video frame is present) displayed on a map.

According to embodiments of the present invention, there may be provided the artificial intelligence diagnosis supporting system that enables the real-time detection of gastrointestinal diseases such as polyps, ulcers, and cancers in various classes based on gastroscopic data.

According to embodiments of the present invention, there may be provided the artificial intelligence diagnosis supporting system that checks an examined area and an unexamined area by tracking the anatomical structures of the gastric walls based on gastroscopic data.

According to embodiments of the present invention, there may be provided the artificial intelligence diagnosis supporting system that classifies the anatomical position of each lesion into the cardia, the fundus, the body, the angle, the antrum, the pylorus and an anterior wall (AW), a posterior wall (PW), a Lesser Curvature (LC), and a Greater Curvature (GC) based on gastroscopic data and then displays an endoscopic position map via a user interface.

According to embodiments of the present invention, there may be provided the artificial intelligence diagnosis supporting system that can present the gastric anatomical position of a region currently being examined in real time based on gastroscopic data.

According to embodiments of the present invention, there may be provided the artificial intelligence diagnosis supporting system that classifies an examined region, presented through artificial intelligence, into the cardia, the fundus, the body, the angle, the antrum, and the pylorus and then displays an endoscopic position map via a user interface so that a blind spot can be displayed via the user interface without being missed.

According to embodiments of the present invention, the sequence in which the gastric walls were passed through may be displayed through the user interface after the termination of examination.

According to embodiments of the present invention, numbers may be assigned to and sequentially display the paths through which an endoscope entered and exited by using a stomach-shaped structure (a position map).

According to embodiments of the present invention, statistical information about the structure (position) of the stomach where an image inferred by artificial intelligence as a lesion risk region was detected.

According to the present invention, work efficiency and diagnostic accuracy may be increased by training on polyps, ulcers, various gastric diseases, etc., which may be missed by a user, based on artificial intelligence medical image diagnosis results for each real-time video frame of a gastroscopic image via the artificial intelligence algorithm and then applying the results of the training to the artificial intelligence diagnosis assisting system.

According to the present invention, there is an effect of preventing in advance a situation that may develop into cancer by detecting a lesion or the like at its early stage. Not only lesions of various sizes but also the locations of the lesions in gastroscopic paths are included in labels and used as learning data. Accordingly, according to the present invention, lesion detection rate may be increased by automatically detecting even a considerably small lesion that may easily be missed by a user, and also the locations of lesions in gastroscopic paths may be extracted.

According to the present invention, the incidence of gastric cancer may be reduced by increasing lesion detection rate and also eliminating gastric cancer risk factors in their early stages. Furthermore, the contributions may be made to reducing the causes of gastric cancer and reducing the frequency of examinations by enabling doctors to find and treat more lesions than before.

According to the present invention, a disease that may easily be missed by a user may be automatically detected during gastroscopy and the location of the disease in a gastric path (a gastroscopic path) may be presented, so that the user may easily check the disease in real time during gastroscopy and even a report adapted to enable other examiners to check it later may be generated through a simple operation.

According to the present invention, there may be provided the optimized content of artificial intelligence medical image diagnosis results for each real-time image frame of an endoscopic image.

According to the present invention, there may be provided the optimized content of a plurality of artificial intelligence medical image diagnosis results for each real-time image frame.

According to the present invention, there may be provided an optimized combination of a plurality of artificial intelligence medical image diagnosis results as display information for each real-time image frame.

According to the present invention, there may be provided an optimized combination of a plurality of artificial intelligence medical image diagnosis results capable of efficiently displaying diagnosis results that are likely to be acquired, are likely to be overlooked, or have a high level of risk in a current image frame.

According to the present invention, there may be provided the user interface and diagnosis computing system that automatically detect and present diagnosis results that are likely to be acquired, are likely to be overlooked, or have a high level of risk in a current image frame, so that medical staff can check and review the diagnosis results in real time during an endoscopy.

According to the present invention, a blind spot, which is a missing part not checked by examination, is displayed to be easily identified by accumulating and displaying the anatomical positions of parts examined by a gastroscope in real time during gastroscopy.

According to the present invention, the adenoma detection rate is increased by detecting various lesions in the stomach regardless of size and color, and a user, who is a medical professional, is helped to easily identify the position of each lesion in the process of performing subsequent examination or reviewing examination results after the completion of examination by also displaying the anatomical position of the corresponding lesion.

According to the present invention, there is provided the user interface that also displays pictures, captured and/or stored as finding information by an examiner during gastroscopy, after the termination of the gastroscopy in accordance with the gastrointestinal anatomical positions thereof, thereby helping a user to search for or access finding information more rapidly and conveniently when making follow-up decisions by using examination results.

The method according to an embodiment of the present disclosure may be implemented as a computer-readable program or code on computer-readable recording media. Computer-readable recording media include all types of recording devices in which data readable by a computer system are stored. The computer-readable recording media may also be distributed in a network-connected computer system to store and execute computer-readable programs or codes in a distributed manner.

The computer-readable recording medium may also include a hardware device specially configured to store and execute program instructions, such as a read only memory (ROM), a random access memory (RAM), and a flash memory. The program instructions may include not only machine language codes such as those generated by a compiler, but also high-level language codes that executable by a computer using an interpreter or the like.

Although some aspects of the present disclosure have been described in the context of an apparatus, it may also represent a description according to a corresponding method, wherein a block or apparatus corresponds to a method step or feature of a method step. Similarly, aspects described in the context of a method may also represent a corresponding block or item or a corresponding device feature. Some or all of the method steps may be performed by (or using) a hardware device, e.g., a microprocessor, a programmable computer, or an electronic circuit. In some embodiments, one or more of the most important method steps may be performed by such an apparatus.

In embodiments, a programmable logic device, e.g., a field programmable gate array, may be used to perform some or all of the functions of the methods described herein. In embodiments, the field programmable gate array may operate in conjunction with a microprocessor to perform one of the methods described herein. In general, the methods are preferably performed by a certain hardware device.

Although described above with reference to the preferred embodiments of the present disclosure, it should be understood that those skilled in the art can variously modify and change the present disclosure within the scope without departing from the spirit and scope of the present disclosure as set forth in the claims below.

What is claimed is:

1. A gastroscopic image diagnosis supporting system for supporting diagnosis of a medical image, the gastroscopic image diagnosis supporting system comprising a computing system,
wherein the computing system comprises:
a reception interface configured to receive a gastroscopic image as the medical image;
memory or a database configured to store one or more medical image analysis models each having a function of analyzing the gastroscopic image; and
a processor,
wherein the processor is configured to:
analyze a video frame of the gastroscopic image using a first medical image analysis model of the medical image analysis models;
classify a gastrointestinal anatomical position of the video frame by identifying a part corresponding to a preset gastrointestinal anatomical position class in the video frame; and
store information about the gastrointestinal anatomical position of the video frame as index information of the finding information together with finding information when a user captures and stores the video frame as the finding information about a gastrointestinal lesion.

2. The gastroscopic image diagnosis supporting system of claim 1, wherein the processor is further configured to:
provide a gastrointestinal anatomical position map;
display the gastrointestinal anatomical position of the video frame on the gastrointestinal anatomical position map and provide the gastrointestinal anatomical position map together with the video frame to the user while the video frame is displayed to the user through a user display; and
display a gastrointestinal anatomical position of the finding information on the gastrointestinal anatomical position map and provide the gastrointestinal anatomical position map to the user when the user requests checking of the finding information after gastroscopy.

3. The gastroscopic image diagnosis supporting system of claim 1, wherein the processor is further configured to:
analyze the video frame of the gastroscopic image by using a second medical image analysis model of the medical image analysis models;
detect whether a region suspected of being a lesion is present in the video frame; and
classify a type of lesion by comparing the region suspected of being a lesion against lesion classes by using a third medical image analysis model of the medical image analysis models.

4. The gastroscopic image diagnosis supporting system of claim 1, wherein the processor is further configured to:
generate gastroscopy entry and exit path information based on the information about the gastrointestinal anatomical position of the video frame and a sequential position at which the video frame is acquired; and
provide the gastroscopy entry and exit path information to the user by displaying the gastroscopy entry and exit path information on a gastrointestinal anatomical position map through a user display.

5. The gastroscopic image diagnosis supporting system of claim 1, wherein the processor is further configured to:
display a user interface for accessing finding information of previous gastroscopy on a first gastrointestinal anatomical position map at a gastrointestinal anatomical position of the finding information of the previous gastroscopy;
provide the first gastrointestinal anatomical position map to the user;
display a path of current gastroscopy and a gastrointestinal anatomical position of a current video frame on a second gastrointestinal anatomical position map; and
provide the second gastrointestinal anatomical position map to the user.

6. A gastroscopic image diagnosis supporting system for supporting diagnosis of a medical image, the gastroscopic image diagnosis supporting system comprising a computing system,
wherein the computing system comprises:
a reception interface configured to receive a gastroscopic image as the medical image;
memory or a database configured to store one or more medical image analysis models each having a function of analyzing the gastroscopic image; and
a processor,
wherein the processor is configured to:
analyze a video frame of the gastroscopic image by using a first medical image analysis model of the medical image analysis models;
classify a gastrointestinal anatomical position of the video frame by identifying a part corresponding to a preset gastrointestinal anatomical position class in the video frame;
display whether a lesion has been found at a position corresponding to the anatomical position class and statistical information about one or more lesions found at the position corresponding to the anatomical position class on a gastrointestinal anatomical position map; and
provide the gastrointestinal anatomical position map to a user through a user display.

7. The gastroscopic image diagnosis supporting system of claim 6, wherein the processor is further configured to:
display whether a lesion has been found at the position corresponding to the anatomical position class and the statistical information about one or more lesions found at the position corresponding to the anatomical position class on a first gastrointestinal anatomical position map by using an analysis result of a video frame of previous gastroscopy; and
display a path of current gastroscopy and a gastrointestinal anatomical position of a current video frame on a second gastrointestinal anatomical position map by using an analysis result of a video frame of the current gastroscopy.

8. The gastroscopic image diagnosis supporting system of claim 7, wherein the processor is further configured to:
display whether a missing anatomical position class is present in the video frame of the previous gastroscopy and an anatomical position of the missing anatomical position class on the first gastrointestinal anatomical position map by using the analysis result of the video frame of the previous gastroscopy.

9. A gastroscopic image diagnosis supporting system for supporting diagnosis of a medical image, the gastroscopic image diagnosis supporting system comprising a computing system,
wherein the computing system comprises:
a reception interface configured to receive a gastroscopic image as the medical image;
memory or a database configured to store one or more medical image analysis models each having a function of analyzing the gastroscopic image; and
a processor,
wherein the processor is configured to:
analyze a video frame of the gastroscopic image using a first medical image analysis model of the medical image analysis models;
classify a gastrointestinal anatomical position of the video frame by identifying a part corresponding to a preset gastrointestinal anatomical position class in the video frame;
generate gastroscopy entry and exit path information based on information about the gastrointestinal anatomical position of the video frame and a sequential position at which the video frame is acquired; and
store the information about the gastrointestinal anatomical position of the video frame and the gastroscopy entry and exit path information as index information of the video frame together with the video frame.

10. The gastroscopic image diagnosis supporting system of claim 9, wherein the processor is further configured to:
provide a gastrointestinal anatomical position map; display a user interface for accessing the video frame on the gastrointestinal anatomical position map at the gastrointestinal anatomical position of the video frame; and
provide the gastrointestinal anatomical position map to a user.

11. The gastroscopic image diagnosis supporting system of claim 9, wherein the processor is further configured to:
provide a gastrointestinal anatomical position map;
display whether the video frame is present at a position corresponding to the anatomical position class, whether a missing anatomical position class is present in the video frame of the gastroscopy, and an anatomical position of the missing anatomical position class on the gastrointestinal anatomical position map; and
provide the gastrointestinal anatomical position map to a user.
